SEQUENCE LISTING (12 sequences)

SEQ ID NO: 1 - protein, 34 aa, synthetic construct
SPHHSGSRHSQSVADTFRRSETSEAWRHPAEHTW

SEQ ID NO: 2 - protein, 50 aa, synthetic construct
CPVCRQRFLLKNLRPNRQLANMVNNLKEISQEAREGTQGERCAVHGERLH

SEQ ID NO: 3 - protein, 50 aa, synthetic construct
YNITDHGSLIYSFSECAFTGPLRPFFSPGFNDGGKNTAPLTLCPLNIGSQ

SEQ ID NO: 4 - protein, 475 aa, Homo sapiens [sequence omitted]

SEQ ID NO: 5 - protein, 398 aa, Homo sapiens
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKEVIIVLERSESWNLKDLDITSPELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 6 - protein, 79 aa, synthetic construct
WKKTVETQKSRIHAEFVQQKNFLVEEEQRQLQELEKDEREQLRILGEKEAKLAQQSQALQELISELDRRCHSSALELLQ

SEQ ID NO: 7 - protein, 286 aa, Homo sapiens
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIRADWKKTVETQKSRIHAEFVQQKNFLVEEEQRQLQELEKDEREQLRILGEKEAKLAQQSQALQELISELDRRCHSSALELLQEVIIVLESPHHSGSRHSQSVADTFRRSETSEAWRHPAEHTWK

SEQ ID NO: 8 - protein, 87 aa, synthetic construct
WKKTVETQKSRIHAEFVQQKNFLVEEEQRQLQELEKDEREQLRILGEKEAKLAQQSQALQELISELDRRCHSSALELLQEVIIVLER

SEQ ID NO: 9 - protein, 109 aa, synthetic construct
EKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQ

SEQ ID NO: 10 - protein, 117 aa, synthetic construct
EKLQVALGELRRKQELAEKLEVEIAIKRADWKKTVETQKSRIHAEFVQQKNFLVEEEQRQLQELEKDEREQLRILGEKEAKLAQQSQALQELISELDRRCHSSALELLQEVIIVLER

SEQ ID NO: 11 - protein, 208 aa, synthetic construct
ELRSVCHVPGLKKMLRTCAVHITLDPDTANPWLILSEDRRQVRLGDTQQSIPGNEERFDSYPMVLGAQHFHSGKHYWEVDVTGKEAWDLGVCRDSVRRKGHFLLSSKSGFWTIWLWNKQKYEAGTYPQTPLHLQVPPCQVGIFLDYEAGMVSFYNITDHGSLIYSFSECAFTGPLRPFFSPGFNDGGKNTAPLTLCPLNIGSQGSTDY

SEQ ID NO: 12 - protein, 127 aa, synthetic construct
MASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVP

(19) United States
(12) Patent Application Publication
(10) Pub. No.: US 2025/0271425 A1
(43) Pub. Date: Aug. 28, 2025

(54) ALTERNATIVE EXON USAGE IN TRIM21 DETERMINES THE ANTIGENICITY OF RO52/TRIM21 IN SYSTEMIC LUPUS ERYTHEMATOSUS

(71) Applicant: THE JOHNS HOPKINS UNIVERSITY, Baltimore, MD (US)

(72) Inventors: Felipe ANDRADE, Baltimore, MD (US); Eduardo GOMEZ-BANUELOS, Baltimore, MD (US)

(21) Appl. No.: 19/062,099

(22) Filed: Feb. 25, 2025

Related U.S. Application Data
(63) Continuation of application No. PCT/US2023/072433, filed on Aug. 18, 2023.
(60) Provisional application No. 63/373,579, filed on Aug. 26, 2022.

(51) Int. Cl. G01N 33/564

(52) U.S. Cl. CPC G01N 33/564; G01N 2800/104; G01N 2800/52; G01N 2800/56

(57) ABSTRACT

The present disclosure relates to methods of identifying a subject with high or low Systemic Lupus Erythematosus Disease Activity Index (SLEDAI), wherein the subject is known to have Systemic Lupus Erythematosus (SLE), and wherein a subject with a high SLEDAI can be treated and a subject with a low SLEDAI can reduce treatment relative to that before the low SLEDAI determination. The methods herein involve detecting the presence of an antibody binding at least one biomarker selected from Ro52Ex4, Ro52Ex3-4, Ro52γCT, and/or Ro52Nt in a sample using routine techniques known in the art.

### CROSS-REFERENCE TO RELATED APPLICATIONS

This application claims the benefit of U.S. Provisional Application No. 63/373,579, filed Aug. 26, 2022, the contents of which are incorporated herein by reference in their entirety.

### FEDERALLY SPONSORED RESEARCH OR DEVELOPMENT

This invention was made with government support under grant AR069569 awarded by the National Institutes of Health. The government has certain rights in the invention.

### SEQUENCE LISTING

The text of the computer readable sequence listing filed herewith, titled “41214_601_SEQUENCE_LISTING.xml,” created Aug. 18, 2023, having a file size of 13.1 kilobytes, is hereby incorporated by reference in its entirety.

### BACKGROUND

Systemic lupus erythematosus (SLE) is a chronic, recurrent, potentially fatal multisystem inflammatory disorder mainly affecting women. SLE is associated with a large spectrum of autoantibodies. IgG antibodies to more than 100 different antigens including DNA, nucleosomes, histones, viral antigens, transcription factors and more have been reported in different SLE patients (46). Surprisingly, there is no serologic diagnosis of SLE and SLE is diagnosed on the basis of eleven criteria defined by the American College of Rheumatology (ACR). These criteria include malar rash, discoid rash, photosensitivity, oral ulcers, arthritis, serositis, renal disorder, neurologic disorder, hematologic disorder (e.g., leucopenia, lymphopenia, hemolytic anemia or thrombocytopenia), immunologic disorder and antibody abnormalities (particularly anti-nuclear antibodies (ANA) and anti-DNA antibodies) (47). According to these criteria, subjects can be clinically diagnosed with SLE if they meet at least four of the eleven criteria. Nevertheless, SLE is still possible even in cases when less than four criteria are present.

Although the precise pathology of SLE is not clear, it is widely accepted that autoantibodies play an important role. Autoantibodies to DNA are highly heterogeneous with respect to their avidity, immunoglobulin subclass composition, cross-reactivity and complement fixing ability. A number of techniques have been utilized for DNA autoantibodies detection, including immunofluorescent assays (IFA), enzyme-linked immunosorbent assays (ELISAs) and radioimmunoassays (RIA). However, the clinical value of anti-dsDNA antibodies largely depends on the assay principle and analytical variables of the methods used to quantitate and immunologically characterize them.

Flares, or higher active disease, occur in approximately 80% of patients during the course of their disease (48), and generally require the introduction or increase in dose of a variety of potentially toxic therapies. The morbidity and mortality associated with flares can be substantial and is related to organ damage resulting from active SLE per se as well as the adverse effects of corticosteroids and immunosuppressive drugs (49).

One of the most difficult challenges in clinical management of complex autoimmune diseases such as SLE is the accurate and early identification of the disease in a patient and differentiation between patients with higher active disease (flare) and those with non-active disease (in remission). There remains a need for improved diagnostic methods and kits useful in identifying high or low Systemic Lupus Erythematosus Disease Activity Index (SLEDAI) in a subject known to have SLE.

### SUMMARY

In some aspects, a method of identifying a subject with high Systemic Lupus Erythematosus Disease Activity Index (SLEDAI), is described, wherein the subject is known to have Systemic Lupus Erythematosus (SLE), said method comprising:
(a) obtaining at least one biological sample from the subject;
(b) detecting the presence of at least one sample antibody binding to an antigen selected from Ro52Ex4, Ro52Ex3-4, and/or Ro52γCT in the sample; and
(c) diagnosing the subject with high SLEDAI if at least one sample antibody that binds to the antigen is detected in the sample.

In other aspects, method of identifying and treating a subject with high Systemic Lupus Erythematosus Disease Activity Index (SLEDAI), is described, wherein the subject is known to have Systemic Lupus Erythematosus (SLE), said method comprising:
(a) obtaining at least one biological sample from the subject;
(b) detecting the presence of at least one sample antibody binding to an antigen selected from Ro52Ex4, Ro52Ex3-4, and/or Ro52γCT in the sample;
(c) diagnosing the subject with high SLEDAI if at least one sample antibody that binds to the antigen is detected in the sample; and
(d) administering at least one pharmaceutical product or pharmaceutical composition to the subject with a high SLEDAI, said pharmaceutical product or pharmaceutical composition comprising a species selected from the group consisting of steroids, cytotoxic agents, rituximab, ocrelizumab, obinutuzumab, veltuzumab, ofatumumab, inebilizumab, blinatumomab, SAR3419,belimumab, tabalumab, atacicept, sifalimumab, anifrolumab, rontalizumab, IFNα-kinoid (IFN-K), and combinations thereof, to prevent and/or ameliorate medical complications associated with high SLEDAI.

In another aspect, a method of identifying a subject with low SLEDAI, wherein the subject is known to have Systemic Lupus Erythematosus (SLE), is described, said method comprising:
(a) obtaining at least one biological sample from the subject;
(b) detecting the presence of a sample antibody binding to a Ro52Nt antigen in the sample; and
(c) diagnosing the subject with low SLEDAI if the sample antibody that binds to the Ro52Nt antigen is detected in the sample.
   Subjects diagnosed with low SLEDAI can reduce treatment relative to that before the low SLEDAI determination.

In yet another aspect, a method of predicting a subject's risk of developing at least one complication associated with disease activity selected from renal failure, digital gangrene, sepsis, anemia, lymphadenopathy, increased frequency of stroke, and/or features of secondary Cushing's syndrome, is described, wherein the subject is known to have SLE, said method comprising:
(a) obtaining at least one biological sample from the subject;
(b) detecting the presence of at least one sample antibody binding to an antigen selected from Ro52Ex4, Ro52Ex3-4, and/or Ro52γCT in the sample; and
(c) predicting that the subject is at risk of developing at least one complication associated with the disease activity if at least one sample antibody that binds to the antigen is detected in the sample.

In still another aspect, a method of predicting a subject's risk of developing at least one complication associated with disease activity selected from renal failure, digital gangrene, sepsis, anemia, lymphadenopathy, increased frequency of stroke, and/or features of secondary Cushing's syndrome, and preventing and/or treating said complication associated with disease activity, is described, wherein the subject is known to have SLE, said method comprising:
(a) obtaining at least one biological sample from the subject;
(b) detecting the presence of at least one sample antibody binding to an antigen selected from Ro52Ex4, Ro52Ex3-4, and/or Ro52γCT in the sample;
(c) predicting that the subject is at risk of developing at least one complication associated with the disease activity if at least one sample antibody that binds to the antigen is detected in the sample; and
(d) administering at least one pharmaceutical product or pharmaceutical composition to the subject at risk of developing at least one complication associated with disease activity, said pharmaceutical product or pharmaceutical composition comprising a species selected from the group consisting of steroids, cytotoxic agents, rituximab, ocrelizumab, obinutuzumab, veltuzumab, ofatumumab, inebilizumab, blinatumomab, SAR3419, belimumab, tabalumab, atacicept, sifalimumab, anifrolumab, rontalizumab, IFNα-kinoid (IFN-K), and combinations thereof, to prevent and/or treat the complications associated with disease activity.

In yet another aspect, a method of predicting if a subject having SLE is in a steady state is described, said method comprising:
(a) obtaining at least one biological sample from the subject;
(b) detecting the presence of a sample antibody binding to a Ro52Nt antigen in the sample; and
(c) predicting that the subject is in a SLE steady state if the sample antibody that binds to the Ro52Nt antigen is detected in the sample.
   Subjects deemed to be in a steady state can reduce treatment relative to that before the low SLEDAI determination.

In another aspect, a method of determining whether a subject suffering from SLE that has been identified as having a high SLEDAI score and is being treated, is responding to said treatment, said method comprising:
(a) obtaining at least one biological sample from the subject;
(b) detecting the presence of at least one sample antibody binding to an antigen selected from Ro52Ex4, Ro52Ex3-4, and/or Ro52γCT in the sample; and
(c) determining that the subject is not responding to said treatment if at least one sample antibody that binds to the antigen is detected in the sample.
   In some embodiments, the subject that is not responding to treatment is further treated to prevent and/or ameliorate medical complications associated with a high SLEDAI score, wherein said treatment comprises administering at least one pharmaceutical product or pharmaceutical composition comprising a species selected from the group consisting of steroids, cytotoxic agents, rituximab, ocrelizumab, 5binutuzumab,veltuzumab, ofatumumab, inebilizumab, blinatumomab, SAR3419, belimumab, tabalumab, atacicept, sifalimumab, anifrolumab, rontalizumab, IFNα-kinoid (IFN-K), and combinations thereof.

In yet another aspect, an article of manufacture comprising a set of reagents to measure the presence of sample antibodies of at least one antigen in a biological sample is described, wherein the at least one antigen is selected from Ro52Ex4, Ro52Ex3-4, Ro52γCT, and/or Ro52Nt, and wherein the set of reagents are bound to a solid support and specifically bind to the sample antibodies in the biological sample.

In another aspect, a kit comprising:
(a) a solid support comprising at least one antigen immobilized on the surface thereof, wherein the at least one antigen is selected from Ro52Ex4, Ro52Ex3-4, Ro52γCT, and/or Ro52Nt, and wherein at least one antigen specifically binds to an antibody present in the sample;
(b) a conjugate comprising an anti-human antibody and a detectable label, wherein the anti-human antibody specifically binds to the antibody present in the sample; and
(c) instructions for use.

Other aspects, features and advantages of the invention will be more fully apparent from the ensuing disclosure and appended claims.

### BRIEF DESCRIPTION OF THE FIGURES

FIG. 1A. Neutrophils (PMN) and PBMCs from 19 consecutive patients with SLE were analyzed by immunoblotting using antibodies to IFIT3, histone H3 (H3) and β-actin (loading controls). Representative samples from 9 patients are shown.

FIG. 1B. Lysates from SLE neutrophils and PBMCs with low and high activation by IFN based on IFIT3 expression. Histone H3 is shown as loading control.

FIG. 1C. Lysates from SLE neutrophils and PBMCs with low and high activation by IFN based on IFIT3 expression were used to screen 20 SLE sera. Data from five SLE sera recognizing a set of autoantigens overexpressed in SLE neutrophils with high IFIT3 expression are shown.

FIG. 1D. Lysates from SLE neutrophils and PBMCs with low and high activation by IFN based on IFIT3 expression were used to screen commercial antibody D-12 to Ro52.

FIG. 1E. Lysates from SLE neutrophils and PBMCs with low and high activation by IFN based on IFIT3 expression were used to screen commercial antibody 671361-1-Ig to Ro52.

FIG. 1F. Lysates from SLE neutrophils and PBMCs with low and high activation by IFN based on IFIT3 expression were used to screen commercial antibody 121081-1-AP to Ro52.

FIG. 1G. Lysates from SLE neutrophils and PBMCs with low and high activation by IFN based on IFIT3 expression were used to screen commercial antibody TA335782 to Ro52.

FIG. 1H. Lysates from SLE neutrophils and PBMCs with low and high activation by IFN based on IFIT3 expression were used to screen commercial antibody AV38248 to Ro52.

FIG. 2. Two-dimensional mapping of autoantigens overexpressed in SLE neutrophils. Cell lysates from IFN-high SLE neutrophils were resolved by two-dimensional electrophoresis and immunoblotted using SLE patient sera that detected the same pattern of bands from FIG. 1C. Once the antigens of interest were mapped, three spots were sliced from a two-dimensional gel stained with GelCode blue (Thermo Scientific) and analyzed by mass spectrometry (Proteomics Core/Mass Spectrometry Facility, Johns Hopkins School of Medicine). The proteins were identified as Ro52/TRIM21.

FIG. 3A. Schematic representation of the transcripts corresponding to TRIM21 isoforms found in a publicly available RNAseq dataset (GSE1491050) from classical monocytes (cMo), neutrophils (PMN), and T cells from SLE patients (n=24) and healthy controls (HC, n=12) using the ‘new tuxedo’ pipeline. Each solid block represents an exon.

FIG. 3B. Differential expression analyses of TRIM21α between HC and SLE according to cell type. Pairwise comparisons between HC and SLE were done using Wilcoxon's Test. *p<0.05, **p<0.01.

FIG. 3C. Differential expression analyses of TRIM21β between HC and SLE according to cell type. Pairwise comparisons between HC and SLE were done using Wilcoxon's Test. *p<0.05, **p<0.01.

FIG. 3D. Differential expression analyses of TRIM21γ between HC and SLE according to cell type. Pairwise comparisons between HC and SLE were done using Wilcoxon's Test. *p<0.05, **p<0.01.

FIG. 4A. Schematic representation of exon usage and structural domains in the Ro52α isoform. RING, “Really interesting new gene”; BB, B-box domain; CC, coiled-coil; LZ, Leucine-zipper.

FIG. 4B. Schematic representation of exon usage and structural domains in the Ro52β isoform.

FIG. 4C. Schematic representation of exon usage and structural domains in the Ro52γ isoform.

FIG. 4D. SLE sera from FIG. 1C were used to immunoblot cell lysates from HEK293 cells transfected with mock (empty vector, EV) or plasmids expressing Ro52α, Ro52β or Ro52γ.

FIG. 4E. Commercial anti-Ro52 antibodies from FIG. 1D were used to immunoblot cell lysates from HEK293 cells transfected with mock (empty vector, EV) or plasmids expressing Ro52α, Ro52β or Ro52γ.

FIG. 4F. Commercial anti-Ro52 antibodies from FIG. 1E were used to immunoblot cell lysates from HEK293 cells transfected with mock (empty vector, EV) or plasmids expressing Ro52α, Ro52β or Ro52γ.

FIG. 4G. Commercial anti-Ro52 antibodies from FIG. 1F were used to immunoblot cell lysates from HEK293 cells transfected with mock (empty vector, EV) or plasmids expressing Ro52α, Ro52β or Ro52γ.

FIG. 4H. Commercial anti-Ro52 antibodies from FIG. 1G were used to immunoblot cell lysates from HEK293 cells transfected with mock (empty vector, EV) or plasmids expressing Ro52α, Ro52β or Ro52γ.

FIG. 4I. Commercial anti-Ro52 antibodies from FIG. 1H were used to immunoblot cell lysates from HEK293 cells transfected with mock (empty vector, EV) or plasmids expressing Ro52α, Ro52β or Ro52γ.

FIG. 5A. Neutrophils (PMN) from 19 consecutive patients with SLE were analyzed by immunoblotting using antibodies to IFIT3, Ro52 (D-12 mouse monoclonal antibody), histone H3 (H3) and β-actin (loading controls). Representative data from 10 patients are shown.

FIG. 5B. PBMCs from 19 consecutive patients with SLE were analyzed by immunoblotting using antibodies to IFIT3, Ro52 (D-12 mouse monoclonal antibody), histone H3 (H3) and β-actin (loading controls). Representative data from 10 patients are shown.

FIG. 5C. Correlation between the expression of IFIT3 and Ro52 in PMN. The expression of Ro52 and IFIT3 was quantified by densitometry from the corresponding bands in FIGS. 5A and 5B, and the values were fitted to a linear regression model.

FIG. 5D. Correlation between the expression of IFIT3 and Ro52 in PBMCs. The expression of Ro52 and IFIT3 was quantified by densitometry from the corresponding bands in FIGS. 5A and 5B, and the values were fitted to a linear regression model.

FIG. 5E. PBMCs and PMN from 12 healthy controls (Ctrl) and one patient with SLE were analyzed by immunoblotting using antibodies to IFIT3, Ro52 (D-12 mouse monoclonal antibody) and H3 (loading control). Representative data from 6 healthy controls are shown. The SLE samples were included for comparison.

FIG. 6A. Schematic representation showing the regions encoded by TRIM21 exon 4, exons 4-5, exons 3-4, and exons 3-4-5 in Ro52.

FIG. 6B. Recombinant proteins containing the sequence encoded by TRIM21 exon 4, exons 4-5, exons 3-4, and exons 3-4-5 were detected by immunoblotting using SLE sera positive for anti-Ro52Ex4 antibodies. Representative data from 6 sera are shown.

FIG. 6C. Levels of antibodies to Ro52Ex4 in sera from the SPARE cohort (SLE) and healthy controls (HC). Comparisons were done using Student's T test. ****p<0.001.

FIG. 6D. Levels of antibodies to Ro52γCT in sera from the SPARE cohort (SLE) and healthy controls (HC). Comparisons were done using Student's T test. ****p<0.001.

FIG. 6E. Venn diagram depicting the anti-Ro52 antibody intersections (overlap) in 128 SLE patients positive for anti-Ro52Ex4, anti-Ro52γCT, and/or anti-Ro52 ‘classic’ antibodies.

FIG. 6F. Recombinant Ro52α, Ro52β, Ro52γ and the Ro52 exon 3-4 encoded sequence were used to analyze by immunoblotting SLE sera from the none overlapping anti-Ro52 classic' antibodies (n=9) (representative data from 5 sera are shown).

FIG. 6G. Recombinant Ro52α, Ro52β, Ro52γ and the Ro52 exon 3-4 encoded sequence were used to analyze by immunoblotting SLE anti-Ro52Ex4 serum.

FIG. 7A. Clinical and laboratory associations present during the clinical course of SLE according to anti-Ro52 antibody type. Odds ratios (OR) were calculated against SLE patients negative for the corresponding anti-Ro52 autoantibody type using a 2×2 Table. Error bars correspond to 95% confidence interval (CI). Associations were tested using chi-square test or Fisher's exact test. P values were calculated with the Student's t test. *p<0.05, **p<0.01, ***p<0.001.

FIG. 7B. Left panel, effect size (Cohen's D) between positive vs. negative SLE patients for each anti-Ro52 antibody type over clinical variables and disease activity evaluated at time of visit. Right panel, heatmap of −log10 (p values) showing the significant associations among anti-Ro52 autoantibody types and variables obtained at time of visit. P values were calculated with the Student's t test. DBP: Diastolic blood pressure, UrPr: Urinary protein, UrRBC: urinary red blood cells, UrPr/Cr: Urinary protein/creatinine ratio, hsCRP: high-sensitivity c-reactive protein, ESR: erythrocyte sedimentation rate, ACL: anti-cardiolipin antibodies, RVVT: Dilute Russell Viper Venom Time, SLEDAI: Systemic Lupus Erythematosus Disease Activity Index, LAI: Lupus Activity Index.

FIG. 8A. Three-way differentially expressed transcript (DET) analysis between SLE patients positive for anti-Ro52Ex4 (n=95), anti-Ro52Nt (n=9), and anti-Ro52γCT (n=21) antibodies. DET between the three anti-Ro52 antibody types (n=926) are shown in a 3D volcano plot. Significance was calculated using the volcano3D package by combining the results of the F test and pairwise comparisons between anti-Ro52Ex4, anti-Ro52Nt, and anti-Ro52γCT, using a multivariate linear model adjusted by anti-DNA positivity and SLEDAI. Color code denotes significant DET in anti-Ro52Ex4 (red), anti-Ro52γCT (green), anti-Ro52Nt (blue), and overlapping genes between anti-Ro52Ex4 and anti-Ro52γCT (yellow), anti-Ro52Nt and anti-Ro52Ex4 (purple), anti-Ro52γCT and anti-Ro52Nt (light blue).

FIG. 8B. Three-way differentially expressed transcript (DET) analysis between SLE patients positive for anti-Ro52Ex4 (n=95), anti-Ro52Nt (n=9), and anti-Ro52γCT (n=21) antibodies. DET between the three anti-Ro52 antibody types (n=926) are shown in a radial plot. Significance was calculated using the volcano3D package by combining the results of the F test and pairwise comparisons between anti-Ro52Ex4, anti-Ro52Nt, and anti-Ro52γCT, using a multivariate linear model adjusted by anti-DNA positivity and SLEDAI. Color code denotes significant DET in anti-Ro52Ex4 (red), anti-Ro52γCT (green), anti-Ro52Nt (blue), and overlapping genes between anti-Ro52Ex4 and anti-Ro52γCT (yellow), anti-Ro52Nt and anti-Ro52Ex4 (purple), anti-Ro52γCT and anti-Ro52Nt (light blue). Representative genes from the enriched pathways on each DET subset are labeled.

FIG. 8C. Activity of the IFN pathway in anti-Ro52 negative (n=63), and SLE patients positive for anti-Ro52Ex4 (n=95), anti-Ro52Nt (n=9), and anti-Ro52γCT (n=21) antibodies. Pathway activity was calculated using gene set variation (GSVA) score. Comparisons between groups were done using the pairwise Wilcoxon test. *p<0.05, ***p<0.001, ****p<0.0001.

FIG. 9. Heatmap of enriched gene ontology (GO) terms across differentially expressed transcripts associated with anti-Ro52 autoantibody types. The top 100 enriched clusters color by p-value associated with anti-Ro52Ex4, anti-Ro52γCT, and anti-Ro52Nt antibodies are shown. Enrichment analyses were carried out using the multiple-list option from Metascape.org.

FIG. 10A. TRIM21/Ro52 splicing variants in SLE keratinocytes. Schematic representation of the transcripts corresponding to TRIM21 isoforms found in a publicly available RNAseq dataset (GSE124939) of keratinocytes from SLE patients (n=7) and healthy controls (HC, n=7) using the ‘new tuxedo’ pipeline. Each solid block represents an exon.

FIG. 10B. Differential expression analyses of TRIM21α (in HC and SLE keratinocytes according to IFNa2 stimulation. Pairwise comparisons between HC and SLE were done using Wilcoxon's Test. ****p<0.0001.

FIG. 10C. Differential expression analyses of TRIM21δ in HC and SLE keratinocytes according to IFNa2 stimulation. Pairwise comparisons between HC and SLE were done using Wilcoxon's Test. ****p<0.0001.

### DETAILED DESCRIPTION OF THE DISCLOSURE

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Analyte” as used herein refers to any component of a biological sample that is desired to be detected (such as, for example, Ro52Ex4, Ro52Ex3-4, Ro52γCT, and/or Ro52Nt). The term can be used to refer to a single component or a sample or a plurality of components in a sample.

“Antibody” and “antibodies” as used herein refers to monoclonal antibodies, monospecific antibodies (e.g., which can either be monoclonal, or may also be produced by other means than producing them from a common germ cell), bi-specific or multi-specific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al., Trends in Biotechnology 21:484-490 (2014)), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass. For simplicity sake, an antibody against an analyte is frequently referred to herein as being either an “anti-analyte antibody” or merely an “analyte antibody.”

“Antibody fragment” or “antigen-binding fragment” as used interchangeably herein, refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

An “anti-human antibody” (also referred to as a “human-specific antibody”) is a type of species-specific antibody that specifically binds human antibodies. In some embodiments, the anti-human antibody may also be referred to as an “anti-analyte antibody,” which is an antibody that binds to an analyte (e.g., at least one of an anti-Ro52Ex4 antibody, an anti-Ro52Ex3-4 antibody, an anti-Ro52γCT antibody, and/or an anti-Ro52Nt antibody). While the anti-human antibody specifically binds to a human antibody, the source of the anti-human antibody need not be human. Indeed, antibodies that specifically bind to a human antibody may be obtained from a non-human mammal, such as a mouse or a rat. In some embodiments, therefore, the anti-human antibody is a mouse antibody, and preferably a mouse monoclonal antibody. The anti-human antibody may be of any type (e.g., IgG, IgE, IgM, IgD, IgA, and IgY), class (e.g., IgG1, IgG2, IgG3, IgG4, IgA1, and IgA2), or subclass.

The terms “immunogen” and “antigen” are used interchangeably herein and refer to any molecule, compound, or substance that induces an immune response in an animal (e.g., a mammal). An “immune response” can entail, for example, antibody production and/or the activation of immune effector cells. An antigen in the context of the disclosure can comprise any subunit, fragment, or epitope of any proteinaceous or non-proteinaceous (e.g., carbohydrate or lipid) molecule that provokes an immune response in a mammal. By “epitope” is meant a sequence of an antigen that is recognized by an antibody or an antigen receptor. Epitopes also are referred to in the art as “antigenic determinants.” In certain embodiments, an epitope is a region of an antigen that is specifically bound by an antibody. In certain embodiments, an epitope may include chemically active surface groupings of molecules such as amino acids, sugar side chains, phosphoryl, or sulfonyl groups. For example, an epitope can be on a polypeptide, a protein, a hapten, a carbohydrate antigen (such as, but not limited to, glycolipids, glycoproteins or lipopolysaccharides), or a polysaccharide. In certain embodiments, an epitope may have specific three-dimensional structural characteristics (e.g., a “conformational” epitope) and/or specific charge characteristics. An antigen can be a protein or peptide of viral, bacterial, parasitic, fungal, protozoan, prion, cellular, or extracellular origin.

A “biochip” as used herein refers to a solid substrate having a generally planar surface to which an adsorbent is attached. Frequently, the surface of a biochip comprises a plurality of addressable locations, each of which location has the adsorbent bound there. Biochips can be adapted to engage a probe interface, and therefore, function as probes. Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

“Binding protein” is used herein to refer to a monomeric or multimeric protein that binds to and forms a complex with a binding partner, such as, for example, a polypeptide, an antigen, a chemical compound or other molecule, or a substrate of any kind. A binding protein specifically binds a binding partner. Binding proteins include antibodies, as well as antigen-binding fragments thereof and other various forms and derivatives thereof as are known in the art and described herein below, and other molecules comprising one or more antigen-binding domains that bind to an antigen molecule or a particular site (epitope) on the antigen molecule. Accordingly, a binding protein includes, but is not limited to, an antibody a tetrameric immunoglobulin, a monoclonal antibody, a chimeric antibody, a CDR-grafted antibody, a humanized antibody, an affinity matured antibody, and fragments of any such antibodies that retain the ability to bind to an antigen. In other aspects, a binding protein can be an aptamer, such as a nucleic acid, that can selectively bind to a specific target.

“Component,” “components,” or “at least one component,” refer generally to capture molecules (e.g., a capture antigen or antibody), a detection molecule or conjugate, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, etc., that can be included in a kit for assay of a test sample, such as a patient urine, whole blood, serum or plasma sample, in accordance with the methods described herein and other methods known in the art. Some components can be in solution or lyophilized for reconstitution for use in an assay.

The term “conjugate,” as used herein, refers to a complex comprising a specific binding pair member and a detectable label. The specific binding pair member of the conjugate, e.g., an anti-human antibody, specifically binds to an analyte (e.g., at least one of an anti-Ro52Ex4 antibody, an anti-Ro52Ex3-4 antibody, an anti-Ro52γCT antibody, and/or an anti-Ro52Nt antibody) present in the sample, which results in the linkage of the conjugate to the captured analyte and formation of an immunosandwich (also referred to herein as an “immunosandwich complex”).

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like). As used herein, a “control subject” relates to a subject or subjects that has does not have SLE.

“Derivative” of an antibody as used herein may refer to an antibody having one or more modifications to its amino acid sequence when compared to a genuine or parent antibody and exhibit a modified domain structure. The derivative may still be able to adopt the typical domain configuration found in native antibodies, as well as an amino acid sequence, which is able to bind to targets (antigens) with specificity. Typical examples of antibody derivatives are antibodies coupled to other polypeptides, rearranged antibody domains, or fragments of antibodies. The derivative may also comprise at least one further compound, e.g., a protein domain, said protein domain being linked by covalent or non-covalent bonds. The linkage can be based on genetic fusion according to the methods known in the art. The additional domain present in the fusion protein comprising the antibody may preferably be linked by a flexible linker, advantageously a peptide linker, wherein said peptide linker comprises plural, hydrophilic, peptide-bonded amino acids of a length sufficient to span the distance between the C-terminal end of the further protein domain and the N-terminal end of the antibody or vice versa. The antibody may be linked to an effector molecule having a conformation suitable for biological activity or selective binding to a solid support, a biologically active substance (e.g., a cytokine or growth hormone), a chemical agent, a peptide, a protein, or a drug, for example.

“Detecting the presence of [biomarker]” or “the presence of [biomarker] is determined or detected” as used herein refers to the qualitative measurement of one or more compounds or biomarkers (e.g., Ro52Ex4, Ro52Ex3-4, Ro52γCT, and/or Ro52Nt) in a biological sample obtained from a subject.

“Identical” or “identity,” as used herein in the context of two or more polypeptide or polynucleotide sequences, can mean that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation.

“Label” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling. Any suitable detectable label as is known in the art can be used. For example, the detectable label can be a radioactive label (such as 3H, 14C, 32P, 33P, 35S, 90Y, 99Tc, 111In, 125I, 131I, 177Lu, 166Ho, and 153Sm), an enzymatic label (such as horseradish peroxidase, alkaline peroxidase, glucose 6-phosphate dehydrogenase, and the like), a chemiluminescent label (such as acridinium esters, thioesters, or sulfonamides; luminol, isoluminol, phenanthridinium esters, and the like), a fluorescent label (such as fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oregon.

As used herein, “marker” refers to a polypeptide (of a particular apparent molecular weight), which is differentially present in a sample taken from patients having SLE as compared to a comparable sample taken from control subjects. The term “biomarker” is used interchangeably with the term “marker.”

“Reference level” as used herein refers to an assay cutoff value (or level) that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). As used herein, the term “cutoff” refers to a limit (e.g., such as a number) above which there is a certain or specific clinical outcome and below which there is a different certain or specific clinical outcome.

“Sample,” “test sample,” “specimen,” “sample from a subject,” “biological sample,” and “patient sample” may be used interchangeably herein to refer to a sample of blood, such as whole blood (including for example, capillary blood, venous blood, dried blood spot, etc.), saliva, tissue, urine, serum, plasma, tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Solid phase” or “solid support” as used interchangeably herein, refers to any material that can be used to attach and/or attract and immobilize (1) one or more capture agents or capture specific binding partners, or (2) one or more detection agents or detection specific binding partners. The solid phase can be chosen for its intrinsic ability to attract and immobilize a capture agent. Alternatively, the solid phase can have affixed thereto a linking agent that has the ability to attract and immobilize the (1) capture agent or capture specific binding partner, or (2) detection agent or detection specific binding partner. For example, the linking agent can include a charged substance that is oppositely charged with respect to the capture agent (e.g., capture specific binding partner) or detection agent (e.g., detection specific binding partner) itself or to a charged substance conjugated to the (1) capture agent or capture specific binding partner, or (2) detection agent or detection specific binding partner. In general, the linking agent can be any binding partner (preferably specific) that is immobilized on (attached to) the solid phase and that has the ability to immobilize the (1) capture agent or capture specific binding partner, or (2) detection agent or detection specific binding partner through a binding reaction. The linking agent enables the indirect binding of the capture agent to a solid phase material before the performance of the assay or during the performance of the assay. For examples, the solid phase can be plastic, derivatized plastic, magnetic, or non-magnetic metal, glass or silicon, including, for example, a test tube, microtiter plate or well, stick, bead (including a microbead), microparticle, biochip, and other configurations known to those of ordinary skill in the art.

“Specific binding” or “specifically binding” as used herein may refer to the interaction of an antibody, a protein, or a peptide with a second chemical species, wherein the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

“Specific binding partner” or “Specific binding member,” as used interchangeable herein, is a member of a specific binding pair that exhibit specific binding. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzymes and enzyme inhibitors, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes and fragments thereof, whether isolated or recombinantly produced.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, chimpanzee, etc.) and a human). In some embodiments, the subject may be a human, a non-human primate or a cat. In some embodiments, the subject is a human. In some embodiments, the subject has been previously diagnosed with SLE. In some embodiments, the subject is a human who is suspected of having SLE. In some embodiments, the subject is a human who has been previously diagnosed with SLE and is suspected of having a “flare” or high disease activity. In some embodiments, the subject or patient is not undergoing treatment for SLE. In other embodiments, the subject or patient may be undergoing treatment for SLE.

As used herein, a “system” refers to a plurality of real and/or abstract elements operating together for a common purpose. In some embodiments, a “system” is an integrated assemblage of hardware and/or software elements. In some embodiments, each component of the system interacts with one or more other elements and/or is related to one or more other elements. In some embodiments, a system refers to a combination of components and software for controlling and directing methods.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. In some embodiments, the prevention or amelioration of the severity of a disease prior to affliction refers to administration of a pharmaceutical composition to a subject that is not at the time of administration afflicted with the disease. In some embodiments, the prevention or amelioration of the severity of a disease refers to administration of a pharmaceutical composition to a subject that is at the time of administration afflicted with the disease. “Preventing” also refers to preventing the recurrence of a disease or of one or more symptoms associated with such disease. “Treatment” and “therapeutically,” refer to the act of treating, as “treating” is defined above.

“Variant” is used herein to describe a peptide or polypeptide that differs from a reference peptide or polypeptide in amino acid sequence by the insertion, deletion, or conservative substitution of amino acids, but retains at least one biological activity. Representative examples of “biological activity” include the ability to be bound by a specific antigen or antibody, or to promote an immune response. Variant is also used herein to describe a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree, and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to refer to an antigenically-reactive fragment of an anti-analyte antibody that differs from the corresponding fragment of anti-analyte antibody in amino acid sequence but is still antigenically reactive and can compete with the corresponding fragment of anti-analyte antibody for binding with the analyte. “Variant” also can be used to describe a polypeptide or a fragment thereof that has been differentially processed, such as by proteolysis, phosphorylation, or other post-translational modification, yet retains its antigen reactivity.

As defined herein, a high “systemic lupus erythematosus disease activity index (SLEDAI)” (35) refers to a state wherein reversible inflammatory damage of multiple organs, such as the kidneys, lungs, blood, skin, heart and brain, may be occurring. The inflammatory damage involves autoantibodies and activation of the complement pathway. Patients diagnosed with a high SLEDAI score have a higher probability of experiencing adverse clinical outcomes including, but not limited to, renal failure, digital gangrene, sepsis, anemia, lymphadenopathy, increased frequency of stroke, and/or features of secondary Cushing's syndrome. If diagnosed early, and treated, the inflammatory damage may be reversible and/or any further damage can be mitigated.

As defined herein, a low “systemic lupus erythematosus disease activity index (SLEDAI)” refers to a patient with SLE in a steady state wherein the patient could reduce the amount of treatment, for example, lower the dosage of a pharmaceutical product per day or per week or per month.

### Methods of Identifying Patients with SLE Having High or Low SLEDAI and Treatment of Same

Broadly, the present invention relates to identifying a subset of patients having Systemic Lupus Erythematosus (SLE) with a high systemic lupus erythematosus disease activity index (SLEDAI). SLE patients having a high SLEDAI have a higher probability of experiencing adverse clinical outcomes including, but not limited to, renal failure, digital gangrene, sepsis, anemia, lymphadenopathy, increased frequency of stroke, and/or features of secondary Cushing's syndrome. Patients with a high SLEDAI can be treated immediately to minimize and reverse the adverse clinical outcomes. In some embodiments, the methods described herein can identify a subset of SLE patients with a low SLEDAI. Patients with a low SLEDAI are considered to be in a steady state and said patients could reduce the amount of treatment relative to that before the low SLEDAI determination.

The origin and mechanisms of autoantigen generation in systemic lupus erythematosus (SLE), a disease characterized by sustained interferon (IFN) signaling, are poorly understood. Ro52, also known as tripartite motif-containing protein 21 (TRIM21), is an interferon (IFN)-induced E3 ubiquitin ligase that drives negative feedback regulation during inflammation (1, 2), and has also been identified as a cytosolic antibody receptor involved in the intracellular clearance of antibody-coated viruses, such as adenovirus (3). Initially described as part of the Ro antigenic particle, Ro52 is among the first autoantigens discovered in systemic lupus erythematosus (SLE) (4, 5), a multisystemic autoimmune disease characterized by high titer autoantibodies leading to immune-mediated tissue damage (6, 51). Antibodies to Ro52 are frequently detected before clinical onset in SLE and are found in up to 40% of patients with established disease (1, 7).

Although Ro52 is mainly expressed by immune cells under steady state conditions (2, 8), to date its pathogenic relevance in SLE has been centered on keratinocytes, mainly because of the initial association between antibodies against the Ro particle with photosensitivity and cutaneous lupus (9-12). In particular, the redistribution of Ro52 on the cell surface and apoptotic blebs of keratinocytes in response to ultraviolet radiation is considered the main mechanism related to the immunogenic source of Ro52 in SLE (13, 14).

Unexpectedly, the present inventors discovered Ro52 as a prominent neutrophil autoantigen with multiple structural forms, of which expression is related to in vivo IFN-induced activation in SLE neutrophils. Different to other cell types, SLE neutrophils are enriched with several Ro52 species containing a core sequence encoded by exon-4 in TRIM21 (hereinafter “Ro52Ex4”), which is the main target of anti-Ro52 antibodies and is found in two Ro52 variants, Ro52α and Ro52γ, upregulated in SLE neutrophils. Further analysis of Ro52γ revealed a new subset of autoantibodies against a unique C-terminal domain generated from a frameshift due to the lack of exon-6 in Ro52γ (anti-Ro52γCT). Antibodies to Ro52Ex4 and Ro52γCT distinguish SLE patient subsets characterized by distinct clinical, laboratory, treatment and transcriptional profiles, which are not discerned by the “classic” anti-Ro52 antibodies.

Accordingly, in a first aspect, methods of identifying whether a subject has low or high SLEDAI, wherein the subject is known to have Systemic Lupus Erythematosus (SLE), is described herein. The method includes obtaining at least one biological sample from the subject. Once at least one sample is obtained from the subject, the presence of an antibody binding at least one biomarker selected from Ro52Ex4, Ro52Ex3-4, Ro52γCT, and/or Ro52Nt, is determined or detected in the sample using routine techniques known in the art, e.g., an assay. In some embodiments, an antibody binding the biomarker Ro52Ex4 is determined or detected in the sample. In some embodiments, an antibody binding the biomarker Ro52Ex3-4 is determined or detected in the sample. In some embodiments, an antibody binding the biomarker Ro52γCT is determined or detected in the sample. In some embodiments, an antibody binding the biomarker Ro52Nt is determined or detected in the sample. If an antibody binding at least one biomarker selected from Ro52Ex4, Ro52Ex3-4, and/or Ro52γCT is detected in the sample, the subject has a high SLEDAI, and is at risk of developing or experiencing adverse clinical outcomes including, but not limited to, renal failure, digital gangrene, sepsis, anemia, lymphadenopathy, increased frequency of stroke, and/or features of secondary Cushing's syndrome. In some embodiments, when detecting an antibody binding Ro52Ex4, the subject has a high SLEDAI score if the antibody levels are greater than or equal to about 15 AU/mL. In some embodiments, when detecting an antibody binding Ro52γCT, the subject has a high SLEDAI score if the antibody levels are greater than or equal to about 30 AU/mL. By identifying a subset of subjects with high SLEDAI early, who because of the high SLEDAI are at higher risk of adverse clinical outcomes including, but not limited to, renal failure, digital gangrene, sepsis, anemia, lymphadenopathy, increased frequency of stroke, and/or features of secondary Cushing's syndrome, the subjects can be treated before the adverse clinical outcomes lead to permanent loss of function or death. If an antibody binding the Ro52Nt biomarker is detected in the sample, the subject is considered to have a low SLEDAI and is considered to be in a steady state because antibodies of Ro52Nt are negatively associated with disease activity. Accordingly, a subject with a low SLEDAI can reduce the amount of treatment relative to that before the low SLEDAI determination, e.g., by reducing the dosage of a pharmaceutical product over time.

Once a subject with SLE is identified as having high SLEDAI, the subject can be treated according to routine techniques known in the art. For example, the subject can be treated with steroids (such as a pharmaceutical product or pharmaceutical composition comprising prednisone, dexamethasone, and/or methylprednisolone), cytotoxic agents (such as a pharmaceutical product or pharmaceutical composition comprising azathioprine, mycophenolate mofetil, methotrexate, leflunomide, chlorambucil, and/or cyclophosphamide), at least one pharmaceutical known to target B cells (such as a pharmaceutical product or pharmaceutical composition comprising rituximab, ocrelizumab, obinutuzumab, veltuzumab, ofatumumab, inebilizumab, blinatumomab, SAR3419, belimumab, tabalumab, and/or atacicept), at least one pharmaceutical known to target IFN type-1 or type-α (such as a pharmaceutical product or pharmaceutical composition comprising sifalimumab, anifrolumab, rontalizumab, and/or IFNα-kinoid (IFN-K), or combinations thereof. In some embodiments, the pharmaceutical compositions comprise at least one pharmaceutically acceptable excipient. Advantageously, the method described herein can identify those subjects with SLE that have high SLEDAI and administer these treatments only to these high SLEDAI subjects, rather than administering expensive and often toxic pharmaceuticals to everyone with SLE regardless of whether they are in a steady state or in a high SLEDAI state (i.e., a flare).

In various embodiments, the pharmaceutical product or pharmaceutical composition can be administered by intravenous, intraarterial, intrathecal, intradermal, intracavitary, oral, rectal, intramuscular, subcutaneous, intracisternal, intravaginal, intraperitonial, topical, buccal, and/or nasal routes of administration, as understood by the person skilled in the art.

Once a subject with SLE is identified as having low SLEDAI (equivalent to a SLEDAI value less than 2), the amount of treatment relative to before the low SLEDAI determination can be reduced, e.g., by reducing the dosage of a pharmaceutical product over time. Advantageously, the method described herein can identify those subjects with SLE that have low SLEDAI and reduce treatments only to these low SLEDAI subjects, rather than continuing to administer the higher amount of expensive and often toxic pharmaceuticals to said subject.

In the above method, the subject can be a human. In some embodiments, the subject is known to have SLE. In some embodiments, the subject is a human who is known to have SLE, but it is not known that they are in a high SLEDAI state. In some embodiments, the subject is a human who is known to have SLE and is suspected to be in a high SLEDAI state. In some embodiments, the subject is a human who is known to have SLE, but it is not known that they are in a low SLEDAI state. In some embodiments, the subject is a human who is known to have SLE and it is suspected that they may be in a low SLEDAI state. In some embodiments, the sample obtained from the subject is a whole blood sample, a plasma sample, a serum sample or a urine sample. In some embodiments, the sample is a whole blood sample. In some embodiments, the sample is a serum sample. In some embodiments, the biological sample is a urine sample.

In another aspect, a method of predicting a subject's risk of developing at least one complication associated with disease activity (such as renal failure, digital gangrene, sepsis, anemia, lymphadenopathy, increased frequency of stroke, and/or features of secondary Cushing's syndrome) wherein the subject is known to have SLE, is described, said method comprising obtaining at least one biological sample from the subject and testing the sample to determine or detect the presence of an antibody binding at least one biomarker selected from Ro52Ex4, Ro52Ex3-4, and/or Ro52γCT. If an antibody binding at least one biomarker selected from Ro52Ex4, Ro52Ex3-4, and/or Ro52γCT is detected in the subject, the subject is identified as being in a high SLEDAI state and has a higher risk of developing damage to organs, such as renal failure or digital gangrene, complications such as sepsis, anemia, lymphadenopathy, increased frequency of stroke, and/or features of secondary Cushing's syndrome. Once a subject with SLE is identified as having high SLEDAI, the subject can be treated with at least one pharmaceutical product known to broadly target immune cells including, but not limited to, steroids, cytotoxic agents, at least one pharmaceutical known to target B cells, at least one pharmaceutical known to target IFN type-1 or type-α, or combinations thereof, as described herein.

In some embodiments, the methods described herein can be repeated as needed to continually monitor and/or assess a subject's risk of high SLEDAI. In some embodiments, the methods described herein can be repeated as need to continually monitor and/or assess if a subject is, or remains in, a steady state (e.g., has a low SLEDAI). In other words, there is no limit on the number of times the methods can be performed.

In yet another aspect, the present disclosure relates to a method of determining whether a subject suffering from SLE that has been identified as being in a high SLEDAI activity state and is being treated, is responding to said treatment. The method includes obtaining at least one biological sample from the subject after treatment has started, e.g., after 7 days, after 14 days, after 21 days, after 28 days, after 2 months, after 3 months, after 6 months, after 12 months, etc. Once at least one sample is obtained from the subject, the presence of an antibody binding at least one biomarker selected from Ro52Ex4, Ro52Ex3-4, Ro52γCT and/or Ro52Nt, is determined or detected in the sample using routine techniques known in the art, e.g., an immunoassay. If an antibody binding at least one biomarker selected from Ro52Ex4, Ro52Ex3-4, and/or Ro52γCT is detected in the subject, the subject is identified as still being in a high SLEDAI state and the subject can continue to be treated with the same pharmaceutical product previously administered or can be treated with at least one different, or additional, pharmaceutical product, as described herein. If an antibody binding the biomarker Ro52Nt is detected in the subject, the subject is identified as being in a low SLEDAI state and hence they are considered to be in a steady state and treatment can be reduced relative to that before the low SLEDAI determination.

In some embodiments, an antibody binding at least one biomarker selected from Ro52Ex4, Ro52Ex3-4, Ro52γCT, and/or Ro52Nt is determined or detected by performing or conducting an assay. The type of assay performed or conducted is not critical. Examples of such assays include, but are not limited to: (1) an immunoassay, such as for example, an enzyme immunoassay (EIA), radioimmunoassay (RIA), fluoroimmunoassay (FIA), chemiluminescent immunoassay (CLIA), or counting immunoassay (CIA); (2) an enzyme-linked immunosorbent assay (ELISA), such as a direct ELISA, an indirect ELISA, a sandwich ELISA, or a competitive ELISA; (3) agglutination assay; or (4) a complement fixation assay.

In some embodiments, an antibody binding at least one biomarker selected from Ro52Ex4, Ro52Ex3-4, Ro52γCT, and/or Ro52Nt is determined or detected by performing or conducting an immunoassay. Any suitable immunoassay may be utilized, such as, for example, a sandwich immunoassay (e.g., monoclonal-polyclonal sandwich immunoassays), competitive inhibition immunoassay (e.g., forward and reverse), a competitive binding assay, heterogeneous assay, and capture on the fly assay. Immunoassay components and techniques that may be used in the disclosed methods are further described in, e.g., International Patent Application Publication Nos. WO 2016/161402 and WO 2016/161400.

In some embodiments, the assay can employ or utilize one or more specific binding partners, wherein at least one specific binding partner is used to capture (e.g., a capture molecule) at least one analyte of interest (e.g., an antibody binding at least one biomarker selected from Ro52Ex4, Ro52Ex3-4, Ro52γCT, and/or Ro52Nt). Examples of capture molecules include one or more of the Ro52Ex4, Ro52Ex3-4, Ro52γCT, and/or Ro52Nt antigens. Optionally, or additionally, at least one second specific binding partner which also binds to the analyte of interest (e.g., an antibody binding at least one biomarker selected from Ro52Ex4, Ro52Ex3-4, Ro52γCT, and/or Ro52Nt) and which may be labeled with at least one detectable label, can also be used (e.g., a detection molecule).

In some embodiments, the assay method comprises (a) contacting a sample from the subject with: (i) a solid support comprising at least one antigen immobilized on the surface thereof, wherein the at least one antigen is selected from Ro52Ex4, Ro52Ex3-4, Ro52γCT, and/or Ro52Nt, and wherein the at least one antigen specifically binds to an antibody present in the sample, and (ii) a conjugate comprising an anti-human antibody and a detectable label, wherein the anti-human antibody specifically binds to the antibody present in the sample; and (b) assessing a signal from the detectable label, wherein a signal from the detectable label indicates the presence of an antibody in the sample, wherein the antibody is an antibody selected from an anti-Ro52Ex4 antibody, an anti-Ro52Ex3-4 antibody, an anti-Ro52γCT antibody, and/or an anti-Ro52Nt antibody.

In some embodiments, the immobilized antigens and conjugate can be contacted with the biological sample simultaneously or sequentially, in any order. In particular embodiments, the at least one antigen (e.g., selected from Ro52Ex4, Ro52Ex3-4, Ro52γCT, and/or Ro52Nt) and the conjugate are contacted with the biological sample simultaneously. In other embodiments, the at least one antigen and the conjugate are contacted with the biological sample sequentially, in any order. Whichever format is chosen, in some embodiments, the method entails contacting a biological sample with the at least one antigen immobilized to a solid support under conditions sufficient for binding of the of the at least one antigen to an antibody (e.g., at least one of an anti-Ro52Ex4 antibody, an anti-Ro52Ex3-4 antibody, an anti-Ro52γCT antibody, and/or an anti-Ro52Nt antibody) present in the sample, thereby forming an antigen-antibody complex. The sample is also contacted with the conjugate comprising an anti-human antibody and a detectable label under conditions sufficient for binding of the conjugate to the antigen-antibody complex, thereby forming an antigen-antibody-conjugate complex. The one or more antigen-antibody-conjugate complexes can be detected.

In some embodiments, a protein microarray is used for determination or detection of at least one analyte of interest (e.g., an antibody binding at least one biomarker selected from Ro52Ex4, Ro52Ex3-4, Ro52γCT, and/or Ro52Nt). In this embodiment, one or more specific binding partners (e.g., such as an antigen) are immobilized on a solid support, such as a biochip. A biological sample from a subject having SLE is passed over the solid support. Bound antibodies (i.e., the analyte of interest) of the antigens, are then detected using any technique known in the art.

In some embodiments, the methods described herein comprise displaying the determination (e.g., determining or detecting the presence of an antibody binding at least one biomarker selected from Ro52Ex4, Ro52Ex3-4, Ro52γCT, and/or Ro52Nt in the sample) on at least one instrument. Suitable instruments include, but are not limited to, a point-of-care device, a core laboratory device (e.g., such as an immunoassay analyzer), a clinical chemistry analyzer, a mass spectrometer, etc., that may contain a user interface that can display the determination.

In some embodiments, the instrument contains software to execute one or more tasks. In some embodiments, the instrument contains software to automatically determine the next appropriate step in a method as described herein. For example, the instrument may contain software that determines the presence of, whether levels are not elevated, and/or whether the test needs to be repeated. The software may display this determination, such as on a graphical user interface.

In some embodiments, the instrument stores software that instructs a processor to execute a given task. In some embodiments, the software stores machine readable instructions that instruct a processor to execute a given task. The machine-readable instructions may be one or more executable programs or portion(s) of an executable program for execution by a computer. The programs may be embodied in software stored on a non-transitory computer readable storage medium such as a CD-ROM, a floppy disk, a hard drive, a DVD, a Blu-ray disk, or a memory associated with the processors. Alternatively, the entire programs and/or parts thereof could alternatively be executed by a device other than the processors and/or embodied in firmware or dedicated hardware. Additionally, or alternatively, processes may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine-readable instructions may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine-readable instructions may be fragmented and stored on one or more storage devices and/or computing devices (e.g., servers). The machine-readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine-readable instructions may be stored in multiple parts, which are individually compressed, encrypted, and stored on separate computing devices, wherein the parts when decrypted, decompressed, and combined form a set of executable instructions that implement a program such as that described herein.

In another example, the machine-readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine-readable instructions may need to be configured (e.g., settings stored, data input, network addresses recorded, etc.) before the machine-readable instructions and/or the corresponding program(s) can be executed in whole or in part. Thus, the disclosed machine-readable instructions and/or corresponding program(s) are intended to encompass such machine readable instructions and/or program(s) regardless of the particular format or state of the machine readable instructions and/or program(s) when stored or otherwise at rest or in transit.

The machine-readable instructions described herein can be represented by any past, present, or future instruction language, scripting language, programming language, etc. For example, the machine-readable instructions may be represented using any of the following languages: C, C++, Java, C #, Perl, Python, JavaScript, HyperText Markup Language (HTML), Structured Query Language (SQL), Swift, etc.

The machine-readable instructions may be stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

### Immunoassays

The disclosed methods detect the presence or of at least one antibody (e.g., at least one of an anti-Ro52Ex4 antibody, an anti-Ro52Ex3-4 antibody, an anti-Ro52γCT antibody, and/or an anti-Ro52Nt antibody) present in a biological sample as described herein. The methods may also be adapted in view of other methods for analyzing analytes. Examples of well-known variations include, but are not limited to, immunoassay, competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, single molecule detection assay, lateral flow assay, etc.

In some embodiments, the method of determining or detecting the presence of at least one antibody (e.g., at least one of an anti-Ro52Ex4 antibody, an anti-Ro52Ex3-4 antibody, an anti-Ro52γCT antibody, and/or an anti-Ro52Nt antibody) present in a biological sample comprises using an immunoassay. In an immunoassay, the analyte of interest, namely, at least one of an anti-Ro52Ex4 antibody, an anti-Ro52Ex3-4 antibody, an anti-Ro52γCT antibody, and/or an anti-Ro52Nt antibody, may be analyzed using at least one first specific binding partner (e.g., at least one antigen selected from Ro52Ex4, Ro52Ex3-4, Ro52γCT, and/or Ro52Nt) and a conjugate in an immunoassay. The presence or amount of the analyte can be determined using the binding of the at least one first specific binding partner and at least one conjugate or second specific binding partner to the analyte of interest (e.g., an antibody).

The presence or amount of the analyte present in a biological sample may be readily determined using an immunoassay. For example, in one aspect, one method that can be used is a chemiluminescent microparticle immunoassay. Other methods that can be used include, for example, mass spectrometry, and immunohistochemistry (e.g., with sections from tissue biopsies). Additionally, methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Specific immunological binding of the antigen to an antibody in the sample can be detected via direct labels, such as fluorescent or luminescent tags, metals and radionuclides attached to the antibody or via indirect labels, such as alkaline phosphatase or horseradish peroxidase.

The use of immobilized antigens may be incorporated into the immunoassay. The antigens may be immobilized onto a variety of supports, such as magnetic or chromatographic matrix particles, the surface of an assay plate (such as microtiter wells), pieces of a solid substrate material, and the like. An assay strip can be prepared by coating the antigen in an array on a solid support. This strip can then be dipped into the test sample and processed quickly through washes and detection steps to generate a measurable signal, such as a colored spot.

In some embodiments, a homogeneous format may be used. For example, after the biological sample is obtained from a subject, a mixture is prepared. The mixture contains the test sample being assessed for the analyte, a first specific binding partner (e.g., antigen), and a second specific binding partner (e.g., an anti-human antibody, with or without a detectable label). The order in which the test sample, the first specific binding partner, and the second specific binding partner are added to form the mixture is not critical. The test sample is simultaneously contacted with the first specific binding partner and the second specific binding partner. In some embodiments, the first specific binding partner and any analyte of interest (e.g., antibody) contained in the test sample may form a first specific binding partner-analyte-complex and the second specific binding partner may form a first specific binding partner-analyte of interest-second specific binding partner complex. Moreover, the second specific binding partner is labeled with or contains a detectable label as described above.

In some embodiments, a heterogeneous format may be used. For example, after the biological sample is obtained from a subject, a first mixture is prepared. The mixture contains the biological sample being assessed for the analyte (e.g., an antibody) and a first specific binding partner (e.g., an antigen), wherein the first specific binding partner and any antibody in the biological sample form a first specific binding partner-analyte complex. The order in which the biological sample and the first specific binding partner are added to form the mixture is not critical.

The first specific binding partner (e.g., an antigen) may be immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc, or a chip. In those embodiments where the solid phase is a bead, the bead may be a magnetic bead or a magnetic particle. Magnetic beads/particles may be ferromagnetic, ferrimagnetic, paramagnetic, superparamagnetic or ferrofluidic. Exemplary ferromagnetic materials include Fe, Co, Ni, Gd, Dy, CrO₂, MnAs, MnBi, EuO, and NiO/Fe. Examples of ferrimagnetic materials include NiFe₂O₄, CoFe₂O₄, Fe₃O₄ (or FeO.Fe₂O₃). Beads can have a solid core portion that is magnetic and is surrounded by one or more non-magnetic layers. Alternately, the magnetic portion can be a layer around a non-magnetic core. The solid support on which the first specific binding partner is immobilized may be stored in dry form or in a liquid. The magnetic beads may be subjected to a magnetic field prior to or after contacting with the sample with a magnetic bead on which the first specific binding partner is immobilized.

After the mixture containing the first specific binding partner-analyte complex is formed, any unbound analyte is removed from the complex using any technique known in the art. For example, the unbound analyte can be removed by washing. Desirably, in some instances, the first specific binding partner is present in excess of any analyte present in the test sample, such that all or most analyte that is present in the test sample is bound by the first specific binding partner.

After any unbound analyte is removed, a second specific binding partner (e.g., an anti-human antibody, with or without a detectable label) is added to the mixture to form a first specific binding partner-analyte of interest-second specific binding partner complex. Moreover, the second specific binding partner is labeled with or contains a detectable label as described above.

The use of immobilized antigens may be incorporated into the immunoassay. The antigens may be immobilized onto a variety of supports, such as magnetic or chromatographic matrix particles (such as a magnetic bead), latex particles or modified surface latex particles, polymer or polymer film, plastic or plastic film, planar substrate, the surface of an assay plate (such as microtiter wells), pieces of a solid substrate material, and the like. The antigens or fragments thereof can be bound to the solid support by adsorption, by covalent bonding using a chemical coupling agent or by other means known in the art, provided that such binding does not interfere with the ability of the antigen to bind analyte. An assay strip can be prepared by coating the antigen or plurality of antigens in an array on a solid support. This strip can then be dipped into the test sample and processed quickly through washes and detection steps to generate a measurable signal, such as a colored spot.

### Kits

Provided herein is a kit, which may be used for assaying or assessing a biological sample for the presence of an antibody binding at least one biomarker selected from Ro52Ex4, Ro52Ex3-4, Ro52γCT, and/or Ro52Nt in the sample. The kit comprises reagents to determine or detect the presence of an antibody binding at least one biomarker selected from Ro52Ex4, Ro52Ex3-4, Ro52γCT, and/or Ro52Nt in the sample.

The kit can contain at least one component (e.g., one or more of the antigens Ro52Ex4, Ro52Ex3-4, Ro52γCT, and/or Ro52Nt) for determining or detecting the presence of an antibody binding at least one biomarker selected from Ro52Ex4, Ro52Ex3-4, Ro52γCT, and/or Ro52Nt in the sample and instructions for determining or detecting the presence of an antibody binding at least one biomarker selected from Ro52Ex4, Ro52Ex3-4, Ro52γCT, and/or Ro52Nt in the sample. In some embodiments, the one or more components may be immobilized or bound to a solid support, such as, for example, a biochip array, a microtiter plate, a stick or a bead (e.g., a microbead). Additionally, instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Alternatively or additionally, the kit can comprise a calibrator or control, e.g., purified, and optionally lyophilized antibodies of at least one biomarker selected from Ro52Ex4, Ro52Ex3-4, Ro52γCT, and/or Ro52Nt, and/or at least one container (e.g., tube, microtiter plates or strips, which can be already coated with at least one biomarker selected from Ro52Ex4, Ro52Ex3-4, Ro52γCT, and/or Ro52Nt) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve.

In some embodiments, anti-human antibodies are included in the kit, i.e., second specific binding partners. In some embodiments, conjugates are included in the kit, wherein the anti-human antibodies incorporate a detectable label, such as a fluorophore, radioactive moiety, enzyme, biotin/avidin label, chromophore, chemiluminescent label, or the like, or the kit can include reagents for labeling the anti-human antibodies for detecting the antibodies (e.g., detection antibodies) and/or for labeling analyte or reagents for detecting the analyte. The conjugates, anti-human antibodies, detectable labels, calibrators, and/or controls can be provided in separate containers or pre-dispensed into an appropriate assay format, for example, into microtiter plates.

Optionally, the kit includes quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit optionally are provided in suitable containers as necessary, e.g., a microtiter plate. The kit can further include containers for holding or storing a sample (e.g., a container or cartridge for a urine, whole blood, plasma, or serum sample). Where appropriate, the kit optionally also can contain reaction vessels, mixing vessels, and other components that facilitate the preparation of reagents or the biological sample. The kit can also include one or more instruments for assisting with obtaining a test sample, such as a syringe, pipette, forceps, measured spoon, or the like.

In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLE

### Methods

### Study Design and Participants

The objective of the study was to identify neutrophil autoantigens with unique patterns of expression linked to in vivo activation by IFN in SLE. Peripheral blood mononuclear cells (PBMCs) and neutrophils were purified from 19 consecutive SLE patients. Cells were lysed and boiled immediately after purification in SDS-sample buffer. Sera from 80 healthy controls and 191 SLE patients from the “Study of biological Pathways, Disease Activity and Response markers in patients with Systemic Lupus Erythematosus” (SPARE) (24, 25) cohort were screened for the presence of anti-Ro52 antibodies. SPARE is a prospective observational cohort that has been extensively described previously (24). Briefly, adult patients (age 18 to 75 years-old) who met the definition of SLE per the revised American College of Rheumatology classification criteria were eligible (34). At baseline, the patient's medical history was reviewed, and information on current medications was recorded. Patients were followed-up over a 2-year period. Patients were treated according to standard clinical practice. Disease activity was assessed using the Safety of Estrogens in Lupus Erythematosus: National Assessment (SELENA) version of the Systemic Lupus Erythematosus Disease Activity Index (SLEDAI) (35) and physician global assessment (PGA) (36). C3, C4, anti-dsDNA (Crithidia), complete blood cell count and urinalysis were performed at every visit. Study participants also underwent whole blood gene expression analysis at baseline using the Affymetrix GeneChip HT HG-U133+ (24, 25). All samples were obtained under informed written consent approved by the Johns Hopkins Institutional Review Board.

### Autoantigen Discovery

PBMC and neutrophil lysates from patients with SLE were used to detect IFIT3 (IFN-induced protein with tetratricopeptide repeats 3) by immunoblotting as a marker of IFN activation (20). To identify neutrophil-specific SLE autoantigens linked to IFN activation, cell lysates with low and high IFIT3 expression were pooled according to cell type and IFIT3 expression and screened by immunoblotting sera from 20 consecutive patients with SLE. The identity of autoantigens of interest was further determined by two-dimensional electrophoresis and mass spectrometry (MS), as previously described (37).

### Antibodies

Mouse monoclonal anti-human IFIT3 (H00003437-B01) was purchased from Abnova, mouse monoclonal anti-human Ro52 (clone D-12) from Santa Cruz Biotechnology, rabbit anti-N-terminal TRIM21 polyclonal antibody from Origene (TA335782), rabbit anti-human TRIM21 polyclonal antibody from Proteintech (121081-1-AP), mouse anti-human TRIM21 monoclonal antibody from Proteintech (671361-1-Ig), rabbit anti-C-terminal TRIM21 polyclonal antibody from Sigma(AV381248), mouse anti-human β-actin from Sigma, and rabbit anti-human histone H3 from Abcam.

### Cloning, Production and Expression of Recombinant Ro52 Isoforms and Domains

The coding sequence of Ro52α was amplified from SLE neutrophil cDNA and used as a template to generate Ro52β and Ro52γ by deleting exons 4 and 6, respectively. Exon deletion was performed using the Q5 Site-Directed Mutagenesis Kit (New England Biolabs). All Ro52 isoforms were cloned into pcDNA3.1 and pET28a(+). The sequences spanning exons 4, 4-5, 3-4 and 3-4-5 were amplified by PCR using Ro52α as template and cloned into pET28(+). Recombinant proteins were expressed in E. coli BL21 (DE3) and purified by Ni-NTA affinity chromatography. All recombinant proteins are N-terminal His-tagged. The complete C-terminal domain of Ro52γ (Ro52γCT) was generated as 35mer synthetic peptide (SPHHSGSRHSQSVADTFRRSETSEA WRHPAEHTWK) (SEQ ID NO: 1). HEK-293T cells were transiently transfected with plasmids to express Ro52 isoforms using lipofectamine 2000 (Invitrogen). HEK-293 cells were lysed after 48 hours post-transfection and used for immunoblotting analyses.

### Detection of Antibodies to Ro52, Ro52Ex4 and Ro52γCT

“Classic” anti-Ro52 antibodies were detected by ELISA using a commercial kit from INOVA (QUANTA Lite SS-A 52, Part number 704505). Anti-Ro52Ex4 and anti-Ro52γCT antibodies were measured in serum/plasma by ELISA produced in our laboratory. Briefly, Nunc Maxisorp plates were coated with 4 μg/mL recombinant Ro52 exon 3-4 to detect anti-Ro52Ex4 antibodies or 5 μg/mL of Ro52γCT was covalently attached to Nunc CovaLink NH plates to detect anti-Ro52γCT antibodies. The plates were blocked for one hour with phosphate buffered saline plus 0.1% Tween-20 (PBST) with 3% non-fat milk. Serum/plasma was diluted 1:1000 in PBST 1% non-fat milk and assayed in duplicate using antigen-conjugated plates and plates without antigen for background subtraction. HRP conjugated goat anti-human IgG was used as a secondary antibody (Diluted at 1:10,000 in PBST 1% nonfat milk). Anti-Ro52Ex4 and anti-Ro52γCT antibody arbitrary units were calculated using a standard curve made of a serial diluted serum from a high-titer SLE patient. Anti-Ro52Nt antibodies were detected by immunoblotting using recombinant Ro52α, Ro52β, Ro52γ and the Ro52 exon 3-4 encoded sequence.

### Differential Gene Expression and Enrichment Analyses

Gene expression analysis from the SPARE cohort was previously described (25). CEL files were subjected to Robust Multichip Average (RMA) background correction, and quantile normalization, using the Oligo package (27). To select only expressed genes in whole blood, transcripts that had a raw signal <100 in less than 10% of samples were filtered out with the genefilter R package. All calculations and analyses were performed using R (ver 4.2.1) and Bioconductor (ver 3.15.2) (38). Differentially expressed transcripts (DETs) were analyzed using the R package ‘limma’ using a multivariate linear model adjusted of anti-dsDNA positivity and SLEDAI (39). Gene set enrichment analyses were done using the online platform Metascape.org (40). Three-way differential gene expression analyses were done using the package ‘volcano3D’ (41) by combining the results from the pairwise comparisons between anti-Ro52Ex4, anti-Ro52γCT, and anti-Ro52Nt antibodies, and the F test calculated with the R package ‘limma.’ To annotate the transcriptomes associated to each anti-Ro52 antibody type, gene set enrichment analyses was performed using the online platform Metascape.org (40). Briefly, in order to do the enrichment analyses, the differentially expressed transcripts (DET) were split into lists according to the results of three-way analysis. Then, the lists of genes were uploaded into the Metascape.org platform. An adjusted p-value <0.05 was considered as significant. Activity of the interferon pathways was calculated using gene-set variation analysis (GSVA) with the R package GSVA (29).

### Isoform Analysis in RNAseq Data

To discover new isoforms of the Ro52 antigen, RNAseq data from neutrophils, classical monocytes (cMo), and T-cells from 24 SLE patients and 12 healthy controls from the publicly available data set GSE149050 was reanalyzed (22). Briefly, the fastq files were aligned to the human genome reference build 38 (GRCh38/hg38) using the splicing-aware aligner HISAT2 (42). Sample's BAM files were further processed using StringTie to quantify de novo assembled transcripts (43). Visualization of the de novo assembled Ro52 (TRIM21) transcripts was done using Ballgown (44).

### Statistical Analysis

Comparisons of continuous variables between two groups were done using Student's T test. Fisher's exact test was used for univariate analysis on SPARE cohort variables, the exact 2×2 package in R was used to calculate the p-value, OR, and 95% CI. Effect size (Cohen's D) between anti-Ro52 positive and negative SLE subjects was calculated using the ‘psych’ R package (45). Pairwise comparisons between of pathway activity were done using Wilcoxon's pairwise test. Statistical significance was set at p<0.05. The statistical analyses were carried with the R software version 4.2.1.

### Results

### Neutrophils Express Distinct Forms of Ro52 Linked to IFN Activation in SLE

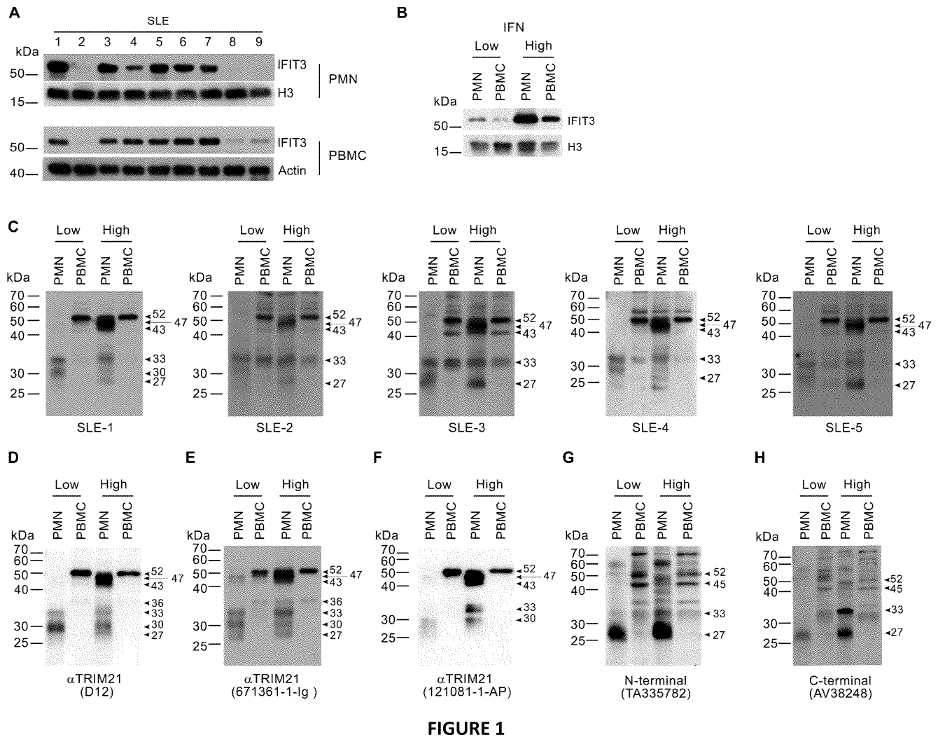

While neutrophils have gained major interest as a source of interferogenic signals in SLE, such as the release of genomic and oxidized mitochondrial DNA that activate plasmacytoid dendritic cells (15-19), it is noteworthy that neutrophils are also important targets of IFN-I in SLE (15). To determine if neutrophil autoantigens can be linked to IFN activation in SLE, in vivo activated cells by IFN were identified through the study of protein expression of the IFN induced protein with tetratricopeptide repeats 3 (IFIT3), as markers of IFN-induced activation (20), in freshly isolated peripheral blood neutrophils and mononuclear cells (PBMCs) from consecutive patients with SLE (see, FIG. 1A).

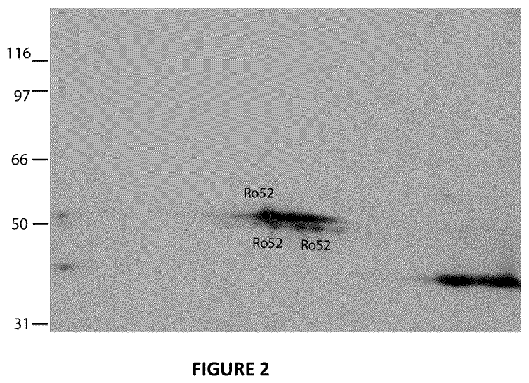

SLE neutrophils and PBMCs with the lowest and highest IFIT3 expression (hereafter IFN low and IFN high, respectively) were then pooled according to cell type and IFIT3 expression (see, FIG. 1B) and used to screen SLE sera (n=20) by immunoblotting to identify neutrophil autoantigens whose expression parallel that of IFIT3. From these studies, the initial focus was on five SLE sera detecting a common pattern of 3 bands of ˜43, 47 and 52 kDa, which were highly expressed by IFN-high SLE neutrophils, but absent in IFN-low SLE neutrophils (see, FIG. 1C). Among these bands, the 52 kDa species was also expressed in PBMCs regardless of IFN status (see, FIG. 1C). Using two-dimensional electrophoresis and mass spectrometry, this set of bands was identified as Ro52 (see, FIG. 2).

Using commercial antibodies, additional studies were performed to determine whether Ro52 has unique patterns of expression in SLE neutrophils and PBMCs according to IFIT3 levels (see, FIGS. 1D-1H). Interestingly, different patterns of Ro52 were detected depending on the commercial antibody utilized. Two mouse monoclonals and one rabbit polyclonal antibody to Ro52, in which the target epitopes were not disclosed (FIGS. 1D and 1E, respectively), reproduced identical patterns of Ro52 detected in neutrophils and PBMCs by SLE serum (i.e., FIG. 1C), including some additional bands of ˜27-33 kDa found in neutrophils. In contrast, two commercial antibodies against the N-terminal and C-terminal regions (amino acid residues 51-100 (SEQ ID NO: 2) and 421-470 (SEQ ID NO: 3), respectively) showed detection of Ro52, albeit modest, in PBCMs and poor detection of the set of Ro52 bands enriched in IFN-high neutrophils (see, FIGS. 1F and 1G). Considering that the antibodies are directed against different regions in Ro52, and without being bound by theory, it is hypothesized that these discrepancies may be explained by the presence of distinct structural forms of Ro52, either transcriptional variants, degradation products, or both.

### SLE Neutrophils Express Novel Splicing Variants of Ro52

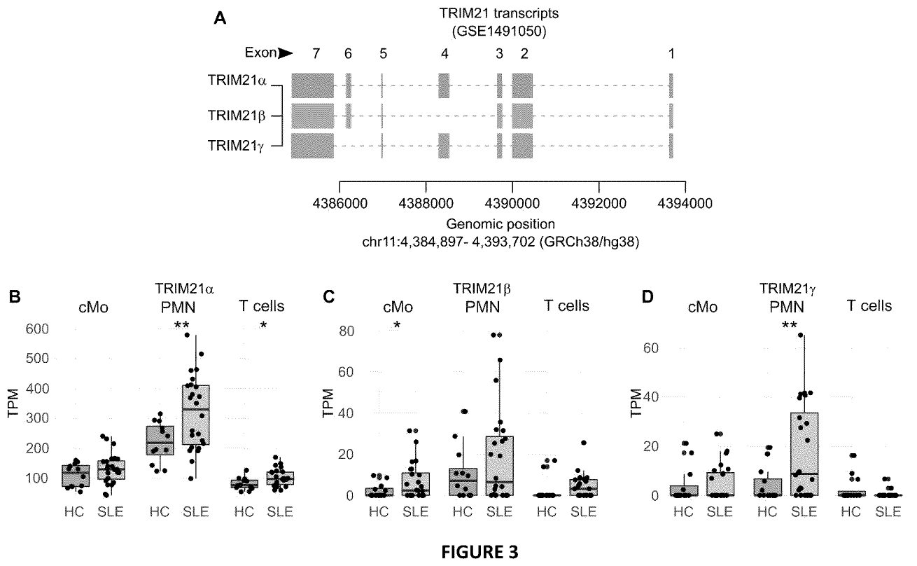

To gain insights into the source of the different Ro52 species found in IFN-high SLE neutrophils, the search for Ro52 splicing variants became the focus. Ro52 is encoded by the TRIM21 gene, which is split into 7 exons (FIG. 3A). The 5′ untranslated sequence is divided between exon 1 and 2, and the initiation codon is located in exon 2 (21). In order to identify novel Ro52 isoforms, a publicly available RNAseq data-set (GSE149050) of three circulating immune cell types from patients with SLE, including neutrophils, classical monocytes (cMo) and T cells (22), was analyzed. Using the ‘new Tuxedo’ pipeline (23), three TRIM21 transcripts were identified: one corresponding to the sequence encoding the full-length protein (termed TRIM21α/Ro52α), a second transcript corresponding to TRIM21β/Ro52β (resulting from the splicing of exon 3 to exon 5, skipping exon 4) (21), and a novel variant termed TRIM21γ/Ro52γ that results from the alternative splicing of exon 5 to exon 7, skipping exon 6 (FIG. 3A).

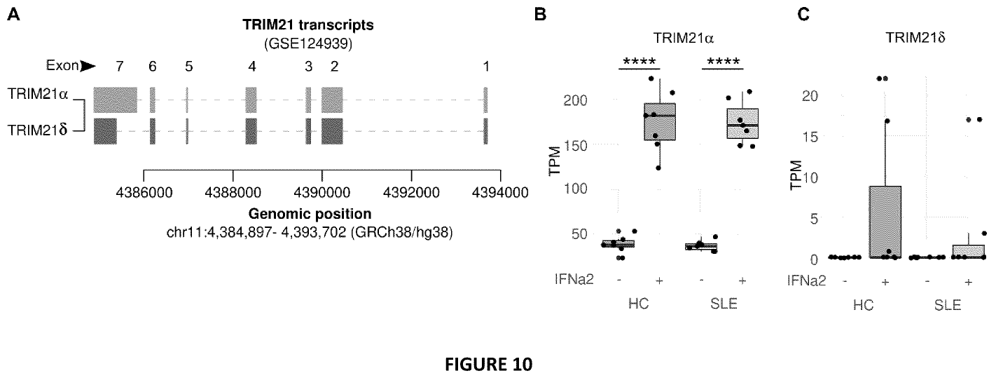

This analysis also demonstrated that the expression of Ro52 variants varies depending on the cell type. Thus, compared to controls, SLE T cells and cMo showed a significant upregulation of the Ro52α and Ro52β transcripts, respectively (FIGS. 3B and 3C, respectively), while SLE neutrophils were distinguished by a significant upregulation of both Ro52α and Ro52γ transcripts (FIGS. 3B and 3D, respectively). In contrast to circulating cells in SLE, keratinocytes only express TRIM21α/Ro52α and a new variant termed TRIM21δ/Ro52δ that results from the alternative splicing of exon 6 to an internal splicing site in exon 7 (FIG. 10A). Interestingly, even though SLE keratinocytes are hyperresponsive to IFNa2 (50), the expression of TRIM21α/Ro52α and TRIM21δ/Ro52δ was not different in SLE compared to healthy control keratinocytes either at baseline or after IFN stimulation (FIGS. 10B and 10C).

### Autoantibodies Against Ro52 Target a Sequence Encoded by Exon 4 in TRIM21

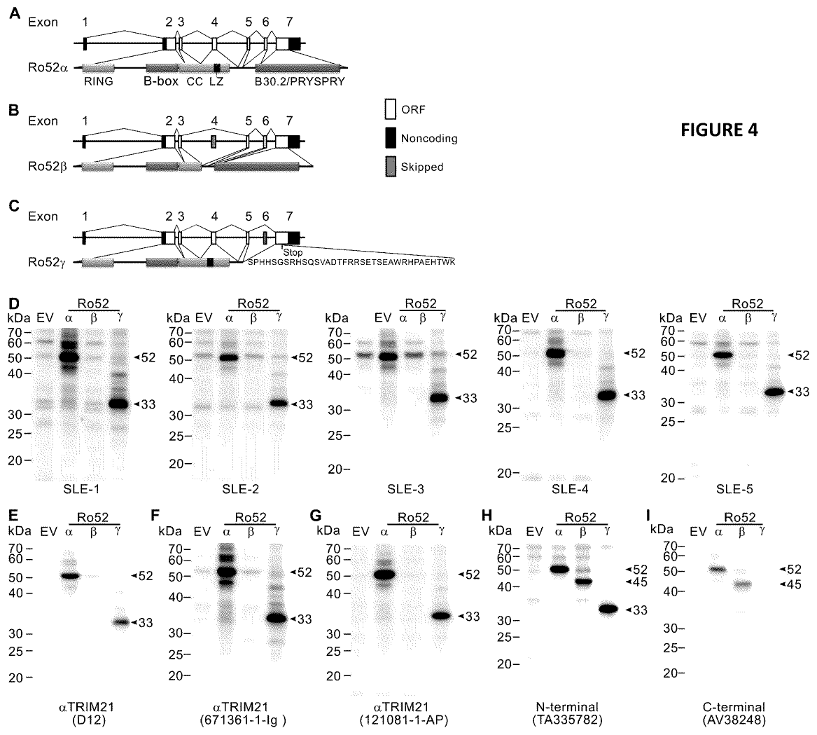

Ro52α (SEQ ID NO: 4), which corresponds to full length Ro52, is a molecule of 52 kDa consisting of 475 amino acid residues (21). The deduced structure contains three distinct domains. The amino-terminal region includes two zinc-finger motifs (a RING-finger and a B-box), followed by a coiled-coil stretch including a putative leucine zipper and a B30.2/PRYSPRY domain in the C-terminal end (FIG. 4A) (1). Ro52β (SEQ ID NO: 5) is a ˜45 kDa protein that lacks the sequence encoded by exon 4 (amino acid residues 167-245 (SEQ ID NO: 6)), which includes the leucine zipper and part of the coiled-coil domain (FIG. 4B) (21). Ro52γ (SEQ ID NO: 7) is a new variant consisting of a C-terminally truncated protein lacking the B30.2/PRYSPRY domain. The predicted product is a protein of 287 amino acids (˜33 kDa) containing the RING, B-box, and coiled-coil domains, followed by a novel C-terminal sequence of 35 amino acid residues generated from a frameshift at the junction of exon 5 and 7 (FIG. 4C).

To investigate whether the distinct patterns of Ro52 detection by SLE sera and commercial antibodies in SLE leukocytes is explained by targeting unique Ro52 isoforms, cell lysates from HEK293 cells transfected to express the different Ro52 variants were immunoblotted. Strikingly, all SLE sera and commercial antibodies recognizing the set of Ro52 bands in IFN-high SLE neutrophils (FIGS. 1C-1F) were specific for Ro52α and Ro52γ (FIGS. 4D-4I), demonstrating that the main epitope targeted by antibodies against the Ro52 neutrophil variants is located within a sequence encoded by exon 4 (hereafter Ro52Ex4), which is missing in Ro52β. Interestingly, since Ro52β contains both the N-terminal and C-terminal domains (FIG. 4B), it is likely that the ˜45 kDa band detected in PBMCs by antibodies to the N-terminal and C-terminal regions of Ro52 (FIGS. 1G and 1H, respectively) corresponds to Ro52β, which is consistent with the upregulation of its transcript in SLE monocytes (FIG. 3C). These data also suggest that Ro52β protein is not detected in neutrophils.

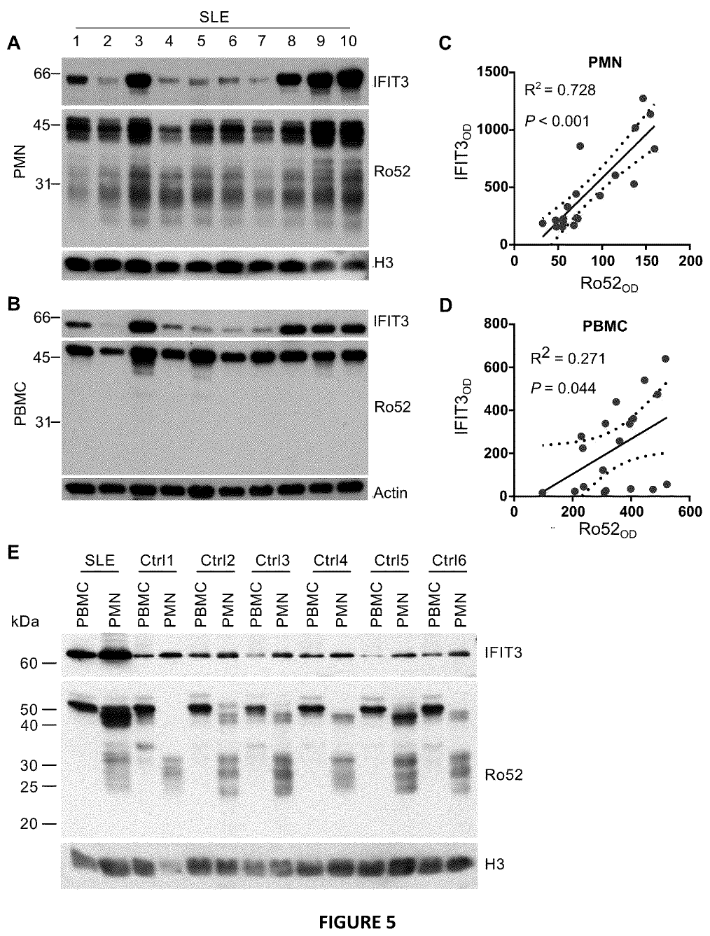

Using the monoclonal antibody D-12 (FIG. 1D), it was further demonstrated that neutrophils from consecutive patients with SLE are highly enriched with Ro52 species containing the Ro52Ex4 epitope, which exhibited a strong correlation with IFIT3 protein expression (R²=0.728, P<0.001) (FIGS. 5A and 5C). In contrast, a single band of Ro52 kDa was detected in SLE PBMCs, which also correlated, albeit less strongly, with IFIT3 levels (R²=0.271, P=0.044) (FIGS. 5B and 5D). In healthy control neutrophils, however, Ro52 was absent or minimally expressed (FIG. 5E). Interestingly, the set of ˜27-33 kDa bands were present in healthy and SLE neutrophils irrespective of IFN activation (FIGS. 1C-1F and FIG. 5E). Taken together, these data demonstrate that SLE neutrophils activated in vivo by IFN are highly enriched with immunogenic Ro52 species which contain the Ro52Ex4 sequence (e.g., Ro52α and Ro52γ), while the Ro52 species are absent in healthy control neutrophils.

### Splicing Variation of Exons 4 and 6 in TRIM21 Determines the Antigenic Targets of Autoantibodies to Ro52 in SLE

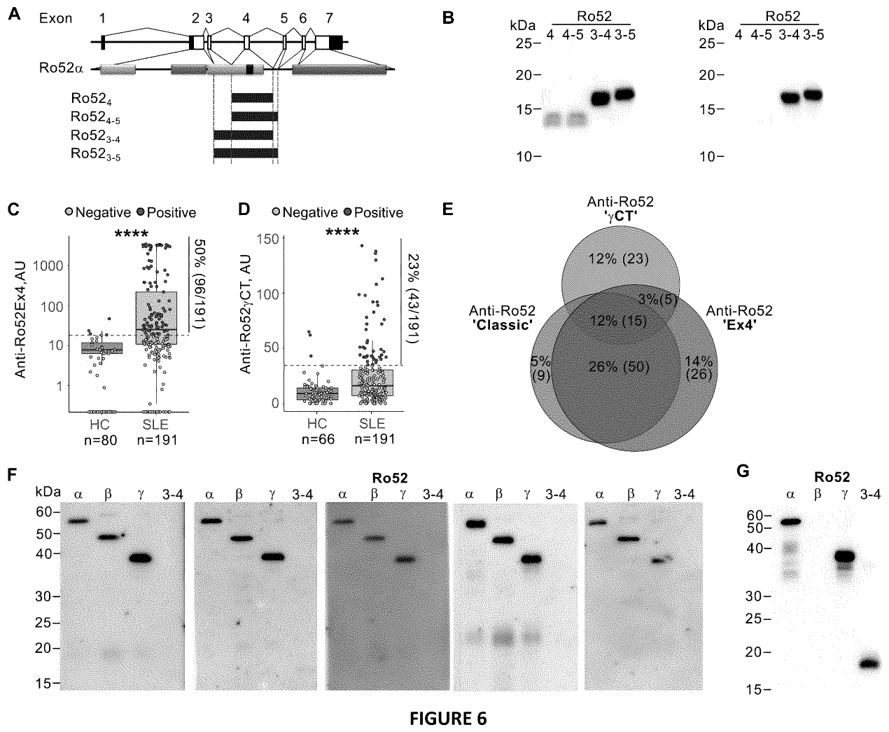

Although Ro52Ex4 is well detected by SLE sera when found in the context of Ro52α and Ro52γ (FIG. 4D), whether the isolated sequence is sufficient for efficient antibody recognition was further addressed. Recombinant proteins containing the Ro52Ex4 amino acid sequence, either alone or in combination with the sequences encoded by flanking exons 3 and/or 5, i.e., exon 4, exons 4-5, exons 3-4, and exons 3-4-5 (amino acid residues 167-245 (SEQ ID NO: 6), 167-253 (SEQ ID NO: 8), 137-245 (SEQ ID NO: 9), and 137-253 (SEQ ID NO: 10), respectively, of Ro52α) (FIG. 6A), were generated, and tested their recognition by SLE sera using immunoblotting (FIG. 6B). Interestingly, although Ro52Ex4 is the target of SLE sera in Ro52α and Ro52γ (FIG. 4D), the presence of exon 3 importantly enhanced or was necessary for the efficient detection of the isolated Ro52Ex4 protein sequence (FIG. 6B). Because SLE sera lack recognition of Ro52β (FIG. 4D), this excludes the possibility that the region encoded by exon 3 is independently immunogenic in SLE. Without being bound by theory, it is likely that the exon 3 encoded sequence facilitates antibody binding by stabilizing the epitope encoded by exon 4, or the sequence encoded at the junction between exons 3 and 4 may play some role in autoantibody recognition.

Since the region encoded by exon 3 enables efficient detection of antibodies to Ro52Ex4 (i.e. anti-Ro52Ex4 antibodies), the recombinant protein containing amino acid residues 137-245 (SEQ ID NO: 9) of Ro52α was used to screen for antibodies in SLE sera from the SPARE lupus cohort, for which extensive clinical and serologic variables are available (24, 25), and 80 healthy controls. Demographic, clinical and laboratory features of the SLE cohort are summarized in Table 1. SLE patients had significantly elevated serum levels of anti-Ro52Ex4 antibodies compared to healthy controls (p<0.0001). Using a cut-off value determined by ROC curve analysis, 50% of SLE patients ( 96/191) were positive for anti-Ro52Ex4 antibodies and 5% of controls ( 4/80) (FIG. 6C).

TABLE 1
Demographic characteristics of SLE patients from SPARE

| Variable | n | n = 190 |
|---|---|---|
| Female Sex | 190 | 177 (93%) |
| Race | 190 | |
| White | | 100 (53%) |
| Black | | 74 (39%) |
| Asian | | 9 (4.7%) |
| Other | | 7 (3.7%) |
| Smoking | 190 | 15 (7.9%) |
| SLEDAI | 187 | 2 (0, 15)¹ |
| Renal SLE | 190 | 99 (52%) |
| Sjogren's Syndrome | 190 | 48 (25%) |
| Anti-DNA | 190 | 123 (65%) |
| Anti-Sm | 189 | 38 (20%) |
| Anti-Ro52 | 190 | 75 (39%) |
| Anti-La | 189 | 28 (15%) |
| Anti-RNP | 189 | 52 (28%) |
| Current treatment | | |
| Prednisone | 190 | 68 (36%) |
| Hydroxicholoroquine | 190 | 167 (88%) |
| Cytotoxic | 190 | 117 (62%) |

¹Median (min-max). Cytotoxic treatment includes: Cyclophosphamide, Mycophenolic acid, Azathioprine, and Methotrexate.

Interestingly, since Ro52Ex4 is found in variants Ro52α and Ro52γ, which are both transcriptionally upregulated in SLE neutrophils (FIGS. 4B and 4D), either isoform could serve as the antigen driving the production of anti-Ro52Ex4 antibodies in SLE. Nevertheless, it is intriguing that autoantibodies against the C-terminal half of Ro52α (amino acid residues 268-475 (SEQ ID NO: 11), also shared by Ro52β) are rare (i.e., 0%) among anti-Ro52 antibodies in SLE (26). While it is possible that the canonical C-terminal region of Ro52α is not a self-immunogen in SLE, it is also possible that the Ro52 variant responsible for the production of antibodies against Ro52Ex4 does not contain the classic C-terminal sequence. Focus was shifted to Ro52γ, which differs from Ro52α in that it possesses a unique C-terminal sequence caused by a frameshift at the junction of exon 5 and 7. Following the same principle that other regions in self-immunogenic Ro52 variants should be targeted by autoantibodies in SLE, antibodies against the unique C-terminal sequence (SEQ ID NO: 1) found in Ro52γ (hereafter Ro52γCT) were sought. Importantly, a BLAST search for this sequence against the catalogue of annotated eukaryotic and prokaryotic proteins showed no similarity to any other known protein. Antibodies to Ro52γCT were significantly increased in SLE compared to healthy controls (p<0.001) and found in 22.5% ( 43/191) of patients with SLE and 3.8% ( 3/80) healthy controls (FIG. 6D).

In the SPARE cohort, 39% ( 74/191) of the patients have antibodies to Ro52 (hereafter anti-Ro52 ‘classic’ antibodies) as detected by the clinically available assay. Analysis of the intersection of the different anti-Ro52 antibodies revealed that 88% ( 65/74) of SLE patients positive for anti-Ro52 ‘classic’ antibodies are also positive for anti-Ro52Ex4 antibodies (FIG. 6E), demonstrating that the majority of anti-Ro52 antibodies in SLE are directed against Ro52Ex4. Further analysis of the small subset of SLE sera positive for anti-Ro52 ‘classic’ antibodies, but negative for antibodies to Ro52Ex4 (n=9), revealed that these sera target the N-terminal sequence (i.e., anti-Ro52Nt) shared by Ro52α, β, and γ (FIG. 6F). This pattern of detection importantly contrasts with anti-Ro52Ex4 serum, which only recognizes Ro52α, Ro52γ and the Ro52Ex3-4 protein sequence (FIG. 6G). Thus, antibodies to biomarker Ro52Nt (SEQ ID NO: 12) represent 4.7% ( 9/191) of anti-Ro52 antibodies in the SLE cohort, which is consistent with a previous study showing that 4% of anti-Ro52 positive SLE sera recognize the N-terminal sequence in Ro52 (amino acid residues 1-127) (26).

### Antibodies Targeting Unique Regions in Ro52 Distinguish Distinct Clinical Subsets and Disease Activity in SLE

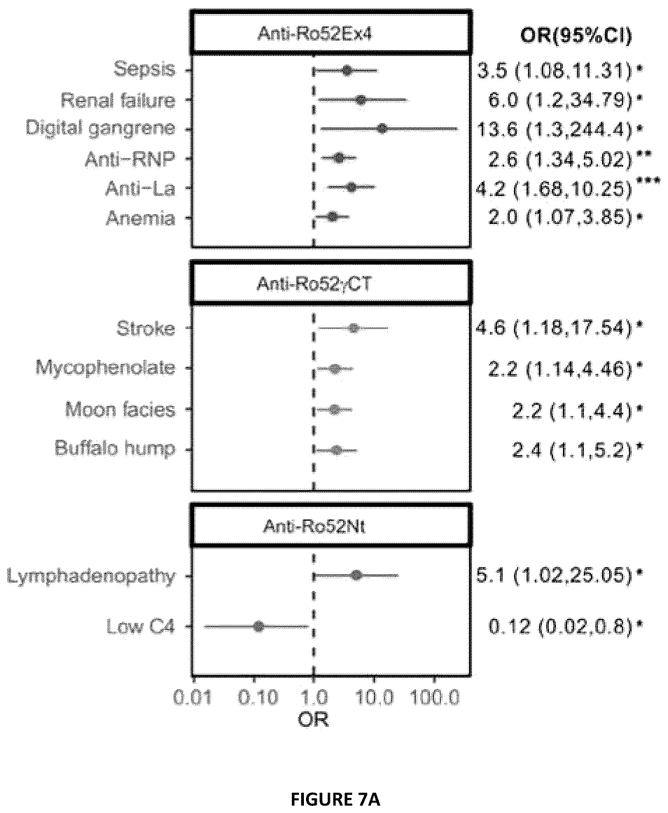

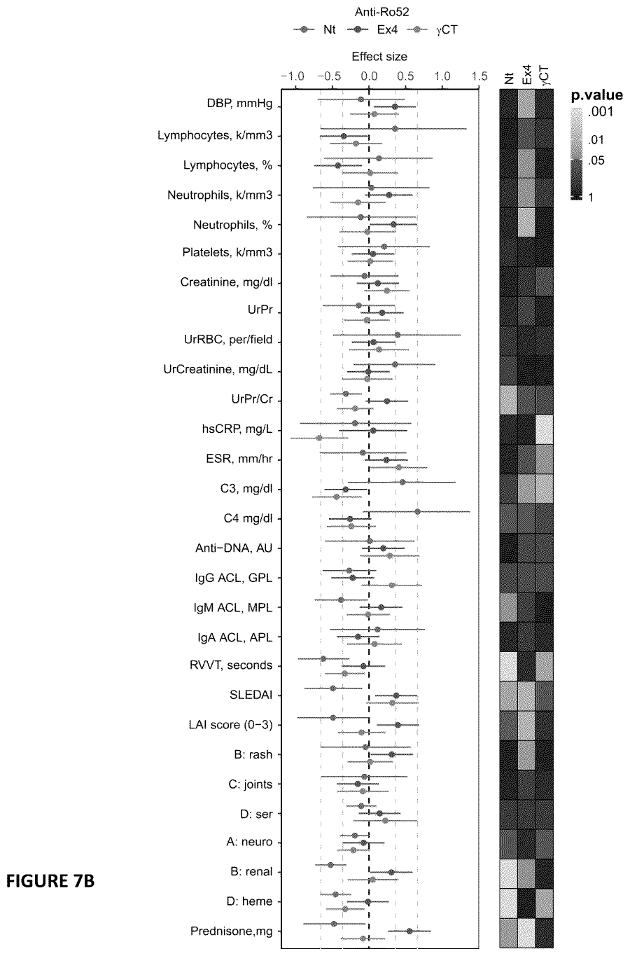

From the analysis of Ro52 expression in SLE neutrophils and further mapping of antibody subsets based on recognition of Ro52 isoforms, antibodies to Ro52 in SLE are classified in three subsets: anti-Ro52Nt, anti-Ro52Ex4 and anti-Ro52γCt antibodies. Interestingly, the distinct antibodies to Ro52 identify clinically relevant endotypes within SLE (FIGS. 7A-7B). Anti-Ro52Nt antibody positivity was associated with lymphadenopathy and interestingly, were less likely to be associated with lower levels of C4 (FIG. 7A). Antibodies to Ro52Ex4 were significantly associated with history of sepsis, renal failure, digital gangrene, anemia, and antibodies to La and RNP (FIG. 7A). In contrast, patients with anti-Ro52γCT antibodies showed an increased frequency of stroke, features of secondary Cushing's syndrome (i.e., moon facies and buffalo hump), and were more likely treated with Mycophenolate (FIG. 7A).

At the time of the visit, the presence of anti-Ro52Nt antibodies showed limited value for SLE disease activity. Rather, these antibodies showed negative associations with laboratory and clinical features linked to disease activity. For instance, this group of patients showed lower SLEDAI, lupus activity index (LAI), renal and hematologic activity, higher C3 and C4, and low use of prednisone (FIG. 7B). In contrast, anti-Ro52Ex4 antibodies were associated with lower lymphocyte and higher neutrophil counts, lower C3, higher SLEDAI and LAI, increased rash and renal activity, and higher prednisone doses (FIG. 7B). Moreover, anti-Ro52γCT antibodies were associated with elevated ERS, lower hsCRP and C3, and a tendency for higher SLEDAI score (FIG. 7B).

Interestingly, despite the prominent overlap with antibodies to Ro52Ex4, anti-Ro52 ‘classic’ antibodies were only associated with anemia, anti-La and sepsis, but not with features of disease activity as anti-Ro52Ex4 antibodies (data not shown). Without being bound by theory, these differences are likely explained because anti-Ro52Nt antibodies, which negatively associate with disease activity, are within the pool of anti-Ro52 ‘classic’ antibodies. Moreover, anti-Ro52Ex4 antibody positivity was found in 26 additional patients negative for anti-Ro52 ‘classic’ antibodies. Thus, autoantibodies targeting specific domains in Ro52 variants appear to be more informative than anti-Ro52 ‘classic’ antibodies to identify clinical subsets in SLE.

### Anti-Ro52 Antibody Subsets Exhibit Distinct Transcriptional Profiles in SLE

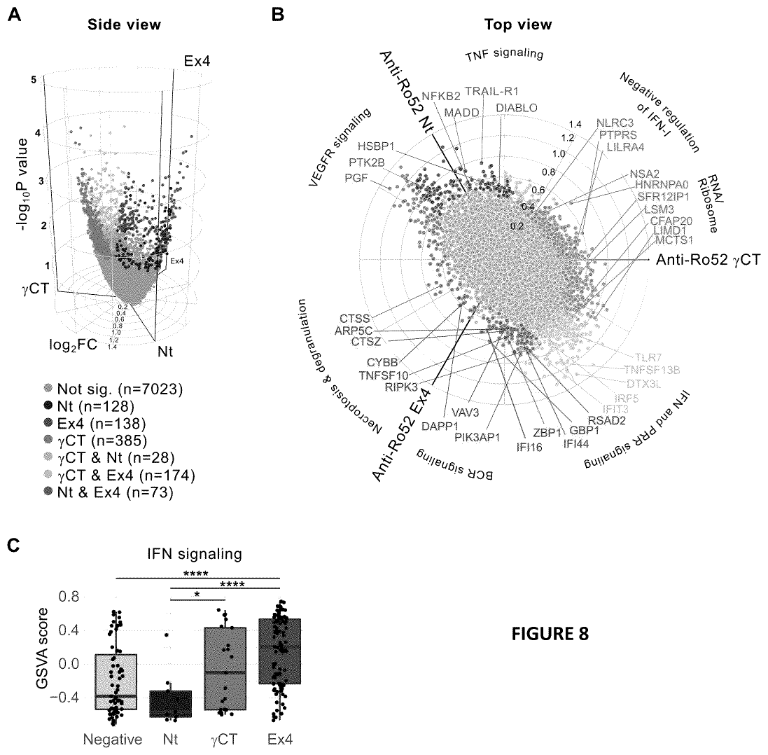

Patients with SLE display unique blood transcriptional signatures associated with immune pathways activated during active disease (27). In particular, it is interesting that the IFN signature has been linked with antibodies to the Ro particle (28). Since anti-Ro52 antibody subsets correlated with distinct clinical features in SLE, the relationship of these antibodies with transcriptional fingerprints activated in SLE was further addressed. Using whole blood gene expression data collected in parallel with the samples used to measure anti-Ro52 antibodies, a three-way comparison between SLE patients positive for antibodies to Ro52Nt, Ro52Ex4 and Ro52γCt was performed (FIG. 8A).

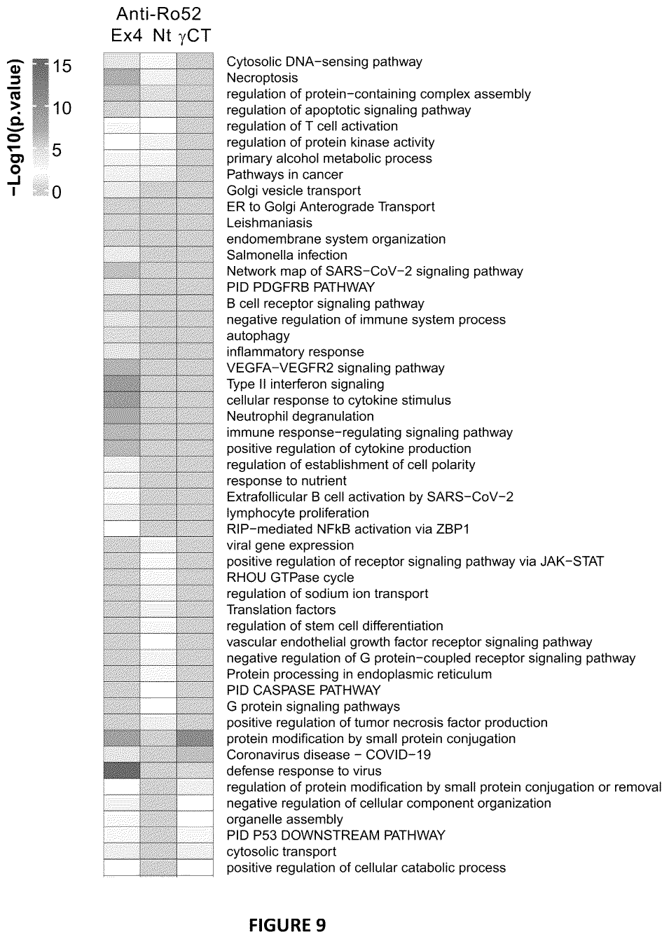

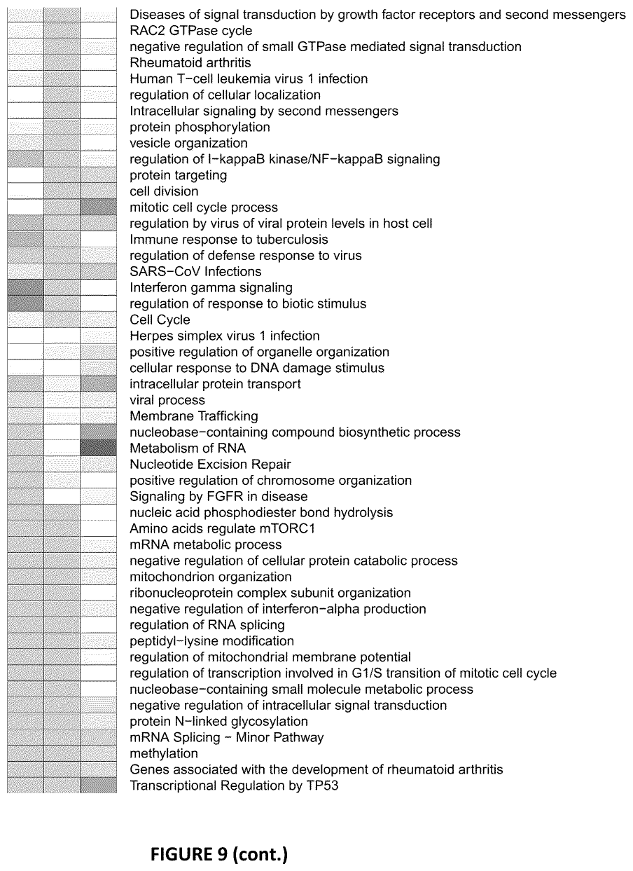

A total of 926 differentially expressed transcripts (DETs) between the three anti-Ro52 antibodies were identified (FIG. 8A). DETs associated with anti-Ro52Ex4 (n=138) were predominantly enriched in genes involved in immune mediated pathways including IFN, intracellular DNA sensing, B-cell receptor signaling, necroptosis and degranulation (FIG. 8B and FIG. 9). Interestingly, a group of common DETs between anti-Ro52γCT and anti-Ro52Ex4 (n=174) was mainly enriched in pathways related to IFN signaling (including increased expression of TNFSF13B, TLR7, IFIT3 and IRF5) and antigen presentation. In contrast, DETs exclusive to anti-Ro52γCT (n=385) contained genes related to p53 signaling, RNA metabolism, ribosome biogenesis, mitochondrial function, and negative-regulation of IFN-I (FIG. 8B and FIG. 9). DETs associated with anti-Ro52Nt antibodies lacked enrichment of IFN-stimulated genes (ISGs), which is consistent with the low disease activity in this small group of patients. Instead, these antibodies were associated with increased vascular endothelial growth factor, TNF signaling and apoptosis (FIG. 8B and FIG. 9). Using gene set variation analysis (GSVA) (29) to quantify the activity of selected pathways on individual SLE patients, it was further confirmed that antibodies to Ro52Ex4 have the most significant association with the IFN signature, followed by anti-Ro52γCT antibodies, and no association of anti-Ro52Nt antibodies with the IFN signature was found (FIG. 8C).

### Discussion

The origin and mechanisms underlying the production of autoantigens in SLE remain unclear. Since core autoantigens in SLE, such as Ro52, Ro60, La, histones, dsDNA and RNPs, are normally expressed under steady state conditions, it has been hypothesized that self-immunogenic forms of these molecules are generated under unique inflammatory environments amplified in SLE. During an examination for autoantigens targeted by SLE serum in in vivo IFN-activated neutrophils from patients with SLE, as described herein, unexpectedly it was found that Ro52 is a prominent IFN-induced autoantigen in SLE neutrophils, which is absent or minimally detected at the protein level in both healthy control and “steady-state” SLE neutrophils. Moreover, IFN-activated SLE neutrophils contained multiple Ro52 species recognized by autoantibodies, which were absent in PBMCs. By combining the analysis of a RNAseq dataset of peripheral blood SLE leukocytes, epitope mapping with commercial anti-Ro52 antibodies, and the study of a large cohort of patients with SLE, it was concluded that the large majority of antibodies to Ro52 in SLE are directed against an epitope encoded by exon 4 in TRIM21 (i.e., Ro52Ex4) found in Ro52α and Ro52γ, which is the major target of anti-Ro52 antibodies in in vivo IFN-activated SLE neutrophils. The significant association of anti-Ro52Ex4 and anti-Ro52γCT antibodies with the IFN signature provides additional evidence that this set of autoantibodies is mechanistically related to the IFN-induced activation in SLE.

Although the expression of Ro52α and Ro52γ can explain the binding of anti-Ro52Ex4 antibodies to SLE neutrophils, the sole expression of these isoforms (molecular weights 52 and ˜33 kDa, respectively) is insufficient to elucidate the origin of the broad range of bands containing the Ro52Ex4 epitope, which are detected in IFN-activated SLE neutrophils. While the existence of additional Ro52 isoforms cannot be ruled out, an alternative hypothesis is that Ro52α and Ro52γ suffer additional modifications, creating complex patterns of Ro52 detection. Without being bound by theory, in the case of Ro52α, cleavage and/or trimming of the N-terminal and C-terminal regions, leaving fragments containing the core Ro52Ex4 sequence intact, could explain the detection of multiple Ro52 species below 52 kDa. Regarding Ro52γ, this isoform would require modifications that increase the molecular weight of the protein. Interestingly, it is noteworthy that both SLE and healthy control neutrophils contain molecular weight bands below 33 kDa, which were detected by the three commercial antibodies against Ro52Ex4, supporting that these bands are likely generated from Ro52α/Ro52γ. As an additional hypothesis, it is possible that Ro52 is normally degraded in neutrophils under steady state conditions, but instead accumulates during SLE disease activity as result of increased expression and likely less degradation, generating neutrophils loaded with large amounts of Ro52, which can become accessible to the immune system as result of neutrophil death. Thus, although neither of these possibilities are exclusive of each other, the most striking finding is that the protein species containing Ro52Ex4 are both enriched in in vivo IFN-activated neutrophils and are the main target of anti-Ro52 antibodies in SLE.

Ro52α is an abundant isoform constitutively expressed by several cell types, such as keratinocytes and immune cells (2, 8, 13). If Ro52α is the main immunogen in SLE, it is intriguing that the major target of anti-Ro52 antibodies is limited to Ro52Ex4, sparing the C-terminal half of the molecule (26). In contrast, however, Ro52γ is targeted both at Ro52Ex4 and the C-terminal domain, offering it an alternative self-antigen to explain the subsets of anti-Ro52 antibodies found in SLE, i.e., anti-Ro52Nt, anti-Ro52Ex4, and anti-Ro52γCT, as well as the limited humoral response against C-terminal Ro52α. The proposal that the N-terminal and C-terminal regions of Ro52α may be cleaved/trimmed, enriching for immunogenic fragments containing Ro52Ex4, is also a potential mechanism to explain the antibody specificity to Ro52Ex4 without targeting the C-terminal domain. Indeed, both models are not exclusive, but complementary. Importantly, whereas it is possible that other cell types in different tissues may generate similar Ro52 patterns as IFN-activated neutrophils in SLE, the experiments presented herein demonstrate that neutrophils are the main cellular source of multiple Ro52 protein species containing Ro52Ex4 in peripheral blood in SLE.

Interestingly, the striking difference in the phenotype of two Ro52/Trim21 knockout mice, in which one develops lupus-like disease and the other has a normal life span (2, 8), has been attributed to the potential production of a truncated Ro52 protein that is overexpressed in the mouse that develops lupus (30, 31). Like Ro52γ, except for the lack of the unique Ro52γ-CT domain, the predicted truncated protein carries the RING domain, B-box and the coiled-coil domain containing the homologous sequence of Ro52Ex4, which is instead encoded by exon 5 in mouse Trim21 (30). In the context of our findings, it is possible that lupus-like disease in these mice is driven by the truncated Ro52γ-like protein (24, 25) which may work either as an autoantigen or through a unique pro-inflammatory function resulting from the lack of the B30.2/PRYSPRY domain, which mediates protein-protein interactions and Fc receptor function (32, 33).

The finding that SLE neutrophils activated by IFN are highly enriched in Ro52 species targeted by the majority of anti-Ro52 antibodies in SLE, underscores neutrophils as the main cellular source of self-immunogenic Ro52 in peripheral blood in SLE. Moreover, the study uncovers the isoform-specific domains Ro52Ex4 and Ro52γCT as the core targets of anti-Ro52 antibodies in SLE, which could be used as potential biomarkers of disease state and to unravel disease mechanisms associated with SLE.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

1. Oke V, and Wahren-Herlenius M. The immunobiology of Ro52 (TRIM21) in autoimmunity: a critical review. J Autoimmun. 2012;39(1-2):77-82.
2. Espinosa A, Dardalhon V, Brauner S, Ambrosi A, Higgs R, Quintana F J, et al. Loss of the lupus autoantigen Ro52/Trim21 induces tissue inflammation and systemic autoimmunity by disregulating the IL-23-Th17 pathway. J Exp Med. 2009;206(8):1661-71.
3. Kiss L, and James L C. The molecular mechanisms that drive intracellular neutralization by the antibody-receptor and RING E3 ligase TRIM21. Semin Cell Dev Biol. 2021.
4. Clark G, Reichlin M, and Tomasi T B, Jr. Characterization of a soluble cytoplasmic antigen reactive with sera from patients with systemic lupus erythmatosus. J Immunol. 1969;102(1):117-22.
5. Ben-Chetrit E, Chan E K L, Sullivan K F, and Tan E M. A 52-kD protein is a novel component of the SS-A/Ro antigenic particle. J Exp Med. 1988;167:1560-71.
6. Rahman A, and Isenberg D A. Systemic lupus erythematosus. N Engl J Med. 2008;358(9):929-39.
7. Heinlen L D, McClain M T, Ritterhouse L L, Bruner B F, Edgerton C C, Keith M P, et al. 60 kD Ro and nRNP A frequently initiate human lupus autoimmunity. PLoS One. 2010;5(3):e9599.
8. Yoshimi R, Chang T H, Wang H, Atsumi T, Morse H C, 3rd, and Ozato K. Gene disruption study reveals a nonredundant role for TRIM21/Ro52 in NF-kappaB-dependent cytokine expression in fibroblasts. J Immunol. 2009;182(12):7527-38.
9. Synkowski D R, Reichlin M, and Provost T T. Serum autoantibodies in systemic lupus erythematosus and correlation with cutaneous features. J Rheumatol. 1982;9(3):380-5.
10. Sontheimer R D, Maddison P J, Reichlin M, Jordon R E, Stastny P, and Gilliam J N. Serologic and HLA associations in subacute cutaneous lupus erythematosus, a clinical subset of lupus erythematosus. Ann Intern Med. 1982;97(5):664-71.
11. Lee L A, Gaither K K, Coulter S N, Norris D A, and Harley J B. Pattern of cutaneous immunoglobulin G deposition in subacute cutaneous lupus erythematosus is reproduced by infusing purified anti-Ro (SSA) autoantibodies into human skin-grafted mice. J Clin Invest. 1989;83(5):1556-62.
12. Davis T L, Lyde C B, Davis B M, and Sontheimer R D. Perturbation of experimental ultraviolet light-induced erythema by passive transfer of serum from subacute cutaneous lupus erythematosus patients. J Invest Dermatol. 1989;92(4):573-7.
13. Casciola-Rosen L A, Anhalt G, and Rosen A. Autoantigens targeted in systemic lupus erythematosus are clustered in two populations of surface structures on apoptotic keratinocytes. J Exp Med. 1994;179:1317-30.
14. Oke V, and Wahren-Herlenius M. Cutaneous lupus erythematosus: clinical aspects and molecular pathogenesis. J Intern Med. 2013;273(6):544-54.
15. Garcia-Romo G S, Caielli S, Vega B, Connolly J, Allantaz F, Xu Z, et al. Netting neutrophils are major inducers of type I IFN production in pediatric systemic lupus erythematosus. Sci Transl Med. 2011;3(73):73ra20.
16. Caielli S, Athale S, Domic B, Murat E, Chandra M, Banchereau R, et al. Oxidized mitochondrial nucleoids released by neutrophils drive type I interferon production in human lupus. J Exp Med. 2016;213(5):697-713.
17. Lood C, Blanco L P, Purmalek M M, Carmona-Rivera C, De Ravin S S, Smith C K, et al. Neutrophil extracellular traps enriched in oxidized mitochondrial DNA are interferogenic and contribute to lupus-like disease. Nat Med. 2016;22(2):146-53.
18. Villanueva E, Yalavarthi S, Berthier C C, Hodgin J B, Khandpur R, Lin A M, et al. Netting neutrophils induce endothelial damage, infiltrate tissues, and expose immunostimulatory molecules in systemic lupus erythematosus. J Immunol. 2011;187(1):538-52.
19. Lande R, Ganguly D, Facchinetti V, Frasca L, Conrad C, Gregorio J, et al. Neutrophils activate plasmacytoid dendritic cells by releasing self-DNA-peptide complexes in systemic lupus erythematosus. Sci Transl Med. 2011;3(73):73ra19.
20. Hall J C, Casciola-Rosen L, Berger A E, Kapsogeorgou E K, Cheadle C, Tzioufas A G, et al. Precise probes of type II interferon activity define the origin of interferon signatures in target tissues in rheumatic diseases. Proc Natl Acad Sci U S A. 2012; 109(43):17609-14.
21. Chan E K, Di Donato F, Hamel J C, Tseng C E, and Buyon J P. 52-kD SS-A/Ro: genomic structure and identification of an alternatively spliced transcript encoding a novel leucine zipper-minus autoantigen expressed in fetal and adult heart. J Exp Med. 1995;182(4):983-92.
22. Panwar B, Schmiedel B J, Liang S, White B, Rodriguez E, Kalunian K, et al. Multi-cell type gene coexpression network analysis reveals coordinated interferon response and cross-cell type correlations in systemic lupus erythematosus. Genome Res. 2021;31(4):659-76.
23. Pertea M, Kim D, Pertea G M, Leek J T, and Salzberg S L. Transcript-level expression analysis of RNA-seq experiments with HISAT, StringTie and Ballgown. Nat Protoc. 2016;11(9):1650-67.
24. Zollars E, Bienkowska J, Czerkowicz J, Allaire N, Ranger A M, Magder L, et al. BAFF (B cell activating factor) transcript level in peripheral blood of patients with SLE is associated with same-day disease activity as well as global activity over the next year. Lupus science & medicine. 2015;2(1):e000063.
25. Zollars E, Courtney S M, Wolf B J, Allaire N, Ranger A, Hardiman G, et al. Clinical Application of a Modular Genomics Technique in Systemic Lupus Erythematosus: Progress towards Precision Medicine. Int J Genomics. 2016;2016:7862962.
26. Infantino M, Meacci F, Grossi V, Benucci M, Morozzi G, Tonutti E, et al. Serological epitope profile of anti-Ro52-positive patients with systemic autoimmune rheumatic diseases. Arthritis Res Ther. 2015;17:365.
27. Banchereau R, Hong S, Cantarel B, Baldwin N, Baisch J, Edens M, et al. Personalized Immunomonitoring Uncovers Molecular Networks that Stratify Lupus Patients. Cell. 2016;165(3):551-65.
28. Kirou K A, Lee C, George S, Louca K, Peterson M G, and Crow M K. Activation of the interferon-alpha pathway identifies a subgroup of systemic lupus erythematosus patients with distinct serologic features and active disease. Arthritis Rheum. 2005;52(5):1491-503.
29. Hanzelmann S, Castelo R, and Guinney J. GSVA: gene set variation analysis for microarray and RNA-seq data. Bmc Bioinformatics. 2013;14:7.
30. Ozato K, Yoshimi R, Chang T H, Wang H, Atsumi T, and Morse H C, 3rd. Comment on “Gene disruption study reveals a nonredundant role for TRIM21/Ro52 in NF-kappa B-dependent cytokine expression in fibroblasts”. J Immunol. 2009;183(12):7619; author reply 720-1.
31. Espinosa A, Brauner S, Ambrosi A, Kuchroo V, and Wahren-Herlenius M. Comment on “Gene Disruption Study Reveals a Nonredundant Role for TRIM21/Ro52 in NF-kappa B-Dependent Cytokine Expression in Fibroblasts Response. Journal of Immunology. 2009;183(12):7620-1.
32. D'Cruz A A, Babon J J, Norton R S, Nicola N A, and Nicholson S E. Structure and function of the SPRY/B30.2 domain proteins involved in innate immunity. Protein Sci. 2013;22(1):1-10.
33. James L C, Keeble A H, Khan Z, Rhodes D A, and Trowsdale J. Structural basis for PRYSPRY-mediated tripartite motif (TRIM) protein function. Proc Natl Acad Sci U S A. 2007;104(15):6200-5.
34. Hochberg M C. Updating the American College of Rheumatology revised criteria for the classification of systemic lupus erythematosus. Arthritis and Rheumatism. 1997;40(9):1725-.
35. Petri M, Kim M Y, Kalunian K C, Grossman J, Hahn B H, Sammaritano L R, et al. Combined oral contraceptives in women with systemic lupus erythematosus. N Engl J Med. 2005;353(24):2550-8.
36. Petri M, Hellmann D, and Hochberg M. Validity and reliability of lupus activity measures in the routine clinic setting. J Rheumatol. 1992;19(1):53-9.
37. Darrah E, Rosen A, Giles J T, and Andrade F. Peptidylarginine deiminase 2, 3 and 4 have distinct specificities against cellular substrates: novel insights into autoantigen selection in rheumatoid arthritis. Ann Rheum Dis. 2012;71(1):92-8.
38. Huber W, Carey V J, Gentleman R, Anders S, Carlson M, Carvalho B S, et al. Orchestrating high-throughput genomic analysis with Bioconductor. Nat Methods. 2015;12(2):115-21.
39. Ritchie M E, Phipson B, Wu D, Hu Y F, Law C W, Shi W, et al. limma powers differential expression analyses for RNA-sequencing and microarray studies. Nucleic Acids Research. 2015;43(7):e47.
40. Zhou Y, Zhou B, Pache L, Chang M, Khodabakhshi A H, Tanaseichuk O, et al. Metascape provides a biologist-oriented resource for the analysis of systems-level datasets. Nat Commun. 2019;10(1):1523.
41. Lewis M J, Barnes M R, Blighe K, Goldmann K, Rana S, Hackney J A, et al. Molecular Portraits of Early Rheumatoid Arthritis Identify Clinical and Treatment Response Phenotypes. Cell Rep. 2019;28(9):2455-70 e5.
42. Kim D, Langmead B, and Salzberg SL. HISAT: a fast spliced aligner with low memory requirements. Nat Methods. 2015;12(4):357-60.
43. Pertea M, Pertea G M, Antonescu C M, Chang T C, Mendell J T, and Salzberg S L. StringTie enables improved reconstruction of a transcriptome from RNA-seq reads. Nat Biotechnol. 2015;33(3):290-5.
44. Fu J, Frazee A C, Collado-Torres L, Jaffe A E, and Leek J T. ballgown: Flexible, isoform-level differential expression analysis. R package version 2.28.0. 2022.
45. Revelle W. psych: Procedures for Psychological, Psychometric, and Personality Research. R package version 2.25. 2022.
46. Sherer Y, Gorstein A, Fritzler M J, Shoenfeld Y, Autoantibody explosion in systemic lupus erythematosus: more than 100 different antibodies found in SLE patients, Semin. Arthritis. Rheum., 2004, 34, 501-37.
47. Hochberg M C, Updating the American College of Rheumatology revised criteria for the classification of systemic lupus erythematosus, Arthritis Rheum., 1997, 40, 1725.
48. Petri M, Perez-Gutthann S, Longenecker J C, Hochberg M, Morbidity of systemic lupus erythematosus: Role of race and socioeconomic status, Am. J. Med., 1991, 91, 345-54.
49. Abu-Shakra M, Urowitz M B, Gladman D D, Gough J, Mortality studies in systemic lupus erythematosus. Results from a single center. I. Causes of death. J Rheumatol., 1995, 22, 1259-64.
50. Tsoi L C, Hile G A, Berthier C C, Sarkar M K, Reed T J, Liu J, et al. Hypersensitive IFN Responses in Lupus Keratinocytes Reveal Key Mechanistic Determinants in Cutaneous Lupus. J Immunol. 2019;202(7):2121-30.
51. Bennett L, Palucka A K, Arce E, Cantrell V, Borvak J, Banchereau J, et al. Interferon and granulopoiesis signatures in systemic lupus erythematosus blood. J Exp Med. 2003;197(6):711-23.

1. A method of identifying a subject with high or low Systemic Lupus Erythematosus Disease Activity Index (SLEDAI), wherein the subject is known to have Systemic Lupus Erythematosus (SLE), said method comprising:
(a) obtaining at least one biological sample from the subject;
(b) detecting the presence of at least one sample antibody binding to an antigen selected from Ro52Ex4, Ro52Ex3-4, Ro52Nt and/or Ro52γCT in the sample; and
(c) (i) diagnosing the subject with high SLEDAI if at least one sample antibody that binds to an antigen selected from Ro52Ex4, Ro52Ex3-4, and/or Ro52γCT is detected in the sample; or
(ii) diagnosing the subject with low SLEDAI if a sample antibody that binds to the Ro52Nt antigen is detected in the sample.

2. The method of claim 1, wherein the biological sample is selected from whole blood, serum, plasma, and/or urine.

3. The method of claim 1, further comprising:
(a) contacting the biological sample from the subject with:
(i) a solid support comprising at least one antigen selected from Ro52Ex4, Ro52Ex3-4, Ro52Nt and/or Ro52γCT immobilized on the surface thereof, wherein the at least one antigen specifically binds to a sample antibody present in the sample; and
(ii) a conjugate comprising an anti-human antibody and a detectable label, wherein the anti-human antibody specifically binds to at least one sample antibody present in the sample; and
(b) assessing a signal from the detectable label, wherein a signal from the detectable label indicates at least one sample antibody is present in the sample.

4. The method of claim 1, wherein the antigen is Ro52Ex4 and wherein the subject has a high SLEDAI score if a sample antibody level greater than or equal to about 15 AU/mL is detected.

5. The method of claim 1, wherein the antigen is Ro52γCT and wherein the subject has a high SLEDAI score if a sample antibody level greater than or equal to about 30 AU/mL is detected.

6. The method of claim 1, further comprising treating the subject diagnosed with high SLEDAI to prevent and/or ameliorate medical complications associated with a high SLEDAI score, wherein said treatment comprises administering at least one pharmaceutical product or pharmaceutical composition comprising a species selected from the group consisting of steroids, cytotoxic agents, rituximab, ocrelizumab, obinutuzumab, veltuzumab, ofatumumab, inebilizumab, blinatumomab, SAR3419, belimumab, tabalumab, atacicept, sifalimumab, anifrolumab, rontalizumab, IFNα-kinoid (IFN-K), and combinations thereof.

7-9. (canceled)

10. The method of claim 1, wherein the subject diagnosed with low SLEDAI can reduce treatment relative to that before the low SLEDAI determination.

11. The method of claim 1, wherein the subject diagnosed with high SLEDAI is at risk of developing at least one complication associated with disease activity selected from renal failure, digital gangrene, sepsis, anemia, lymphadenopathy, increased frequency of stroke, and/or features of secondary Cushing's syndrome.

12-15. (canceled)

16. The method of claim 11, wherein the subject at risk of developing at least one complication associated with disease activity selected from renal failure, digital gangrene, sepsis, anemia, lymphadenopathy, increased frequency of stroke, and/or features of secondary Cushing's syndrome, is treated to prevent and/or ameliorate the complications associated with the disease activity, wherein said treatment comprises administering at least one pharmaceutical product or pharmaceutical composition comprising a species selected from the group consisting of steroids, cytotoxic agents, rituximab, ocrelizumab, obinutuzumab, veltuzumab, ofatumumab, inebilizumab, blinatumomab, SAR3419, belimumab, tabalumab, atacicept, sifalimumab, anifrolumab, rontalizumab, IFNα-kinoid (IFN-K), and combinations thereof.

17. The method of claim 1, wherein the subject diagnosed with low SLEDAI is in a steady state.

18-19. (canceled)

20. The method of claim 17, wherein the subject in a SLE steady state can reduce treatment relative to that before the low SLEDAI determination.

21. The method of claim 6, further comprising determining if the subject identified as having a high SLEDAI score and is being treated is responding to said treatment, said method comprising:
(d) obtaining at least one additional biological sample from the subject;
(e) detecting the presence of at least one sample antibody binding to an antigen selected from Ro52Ex4, Ro52Ex3-4, and/or Ro52γCT in the sample; and
(f) determining that the subject is not responding to said treatment if at least one sample antibody that binds to the antigen selected from Ro52Ex4, Ro52Ex3-4, and/or Ro52γCT is detected in the sample.

22. The method of claim 21, wherein the biological sample is selected from whole blood, serum, plasma, and/or urine.

23-25. (canceled)

26. The method of claim 21, wherein the subject that is not responding to treatment is further treated to prevent and/or ameliorate medical complications associated with a high SLEDAI score, wherein said treatment comprises administering at least one pharmaceutical product or pharmaceutical composition comprising a species selected from the group consisting of steroids, cytotoxic agents, rituximab, ocrelizumab, obinutuzumab, veltuzumab, ofatumumab, inebilizumab, blinatumomab, SAR3419, belimumab, tabalumab, atacicept, sifalimumab, anifrolumab, rontalizumab, IFNα-kinoid (IFN-K), and combinations thereof.

27-28. (canceled)

29. An article of manufacture comprising a set of reagents to measure the presence of sample antibodies of at least one antigen in a biological sample, wherein the at least one antigen is selected from Ro52Ex4, Ro52Ex3-4, Ro52γCT, and/or Ro52Nt, and wherein the set of reagents are bound to a solid support and specifically bind to the sample antibodies in the biological sample.

30. The article of manufacture of claim 29, wherein the reagents are specific binding partners.

31. The article of manufacture of claim 29, further comprising a conjugate comprising an anti-human antibody and a detectable label.

32. The article of manufacture of claim 29, wherein the solid support is a biochip, a microtiter plate, a stick, a bead or any combination thereof.

33. A test kit comprising the article of manufacture of claim 29.

34. A kit comprising:
(a) a solid support comprising at least one antigen immobilized on the surface thereof, wherein the at least one antigen is selected from Ro52Ex4, Ro52Ex3-4, Ro52γCT, and/or Ro52Nt, and wherein at least one antigen specifically binds to an antibody present in the sample;
(b) a conjugate comprising an anti-human antibody and a detectable label, wherein the anti-human antibody specifically binds to the antibody present in the sample; and
(c) instructions for use.
